(19) United States
(12) Patent Application Publication
(10) Pub. No.: US 2014/0377223 A1
(43) Pub. Date: Dec. 25, 2014

(54) SUBSTITUTED BIPHENYLENE COMPOUNDS AND METHODS OF USE THEREOF FOR THE TREATMENT OF VIRAL DISEASES

(71) Applicants: Joseph A. Kozlowski, Princeton, NJ (US); Bandarpalle B. Shankar, Neshanic Station, NJ (US)

(72) Inventors: Joseph A. Kozlowski, Princeton, NJ (US); Bandarpalle B. Shankar, Neshanic Station, NJ (US)

(21) Appl. No.: 13/812,052

(22) PCT Filed: Jul. 21, 2011
(86) PCT No.: PCT/US11/44769

§ 371 Date: Sep. 9, 2014

Related U.S. Application Data
(60) Provisional application No. 61/367,638, filed on Jul. 26, 2010.

(51) Int. Cl. C07D 403/14; A61K 45/06; A61K 31/695; A61K 31/439; C07F 7/08; A61K 31/4178; C07D 401/14

(52) U.S. Cl. CPC C07D 403/14; A61K 31/4178; A61K 45/06; C07D 401/14; A61K 31/439; C07F 7/0816; A61K 31/695
USPC 424/85.7; 548/314.7; 514/397; 514/43; 546/112; 514/299; 548/110; 514/63

(57) ABSTRACT

The present invention relates to novel Substituted Biphenylene Compounds of Formula (I):

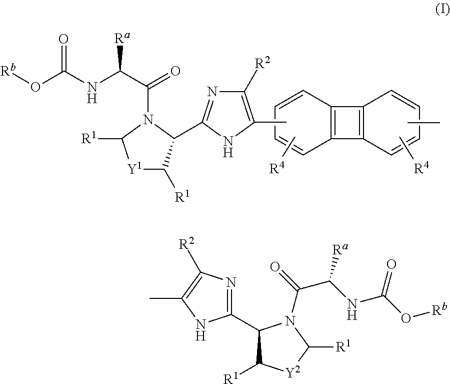

and pharmaceutically acceptable salts thereof, wherein Y¹, Y², R¹, R², R⁴, R^{a} and R^{b} are as defined herein. The present invention also relates to compositions comprising at least one Substituted Biphenylene Compound, and methods of using the Substituted Biphenylene Compounds for treating or preventing HCV infection in a patient.

### FIELD OF THE INVENTION

The present invention relates to novel Substituted Biphenylene Compounds, compositions comprising at least one Substituted Biphenylene Compound, and methods of using the Substituted Biphenylene Compounds for treating or preventing HCV infection in a patient.

### BACKGROUND OF THE INVENTION

Hepatitis C virus (HCV) is a major human pathogen. A substantial fraction of these HCV-infected individuals develop serious progressive liver disease, including cirrhosis and hepatocellular carcinoma, which are often fatal. HCV is a (+)-sense single-stranded enveloped RNA virus that has been implicated as the major causative agent in non-A, non-B hepatitis (NANBH), particularly in blood-associated NANBH (BB-NANBH) (see, International Publication No. WO 89/04669 and European Patent Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

It is well-established that persistent infection of HCV is related to chronic hepatitis, and as such, inhibition of HCV replication is a viable strategy for the prevention of hepatocellular carcinoma. Current therapies for HCV infection include α-interferon monotherapy and combination therapy comprising α-interferon and ribavirin. These therapies have been shown to be effective in some patients with chronic HCV infection, but suffer from poor efficacy and unfavorable side-effects and there are currently efforts directed to the discovery of HCV replication inhibitors that are useful for the treatment and prevention of HCV related disorders.

Current research efforts directed toward the treatment of HCV includes the use of antisense oligonucleotides, free bile acids (such as ursodeoxycholic acid and chenodeoxycholic acid) and conjugated bile acids (such as tauroursodeoxycholic acid). Phosphonoformic acid esters have also been proposed as potentially useful for the treatment of various viral infections, including HCV. Vaccine development, however, has been hampered by the high degree of viral strain heterogeneity and immune evasion and the lack of protection against reinfection, even with the same inoculum.

In light of these treatment hurdles, the development of small-molecule inhibitors directed against specific viral targets has become a major focus of anti-HCV research. The determination of crystal structures for NS3 protease, NS3 RNA helicase, NS5A, and NS5B polymerase, with and without bound ligands, has provided important structural insights useful for the rational design of specific inhibitors.

Recent attention has been focused toward the identification of inhibitors of HCV NS5A. HCV NS5A is a 447 amino acid phosphoprotein which lacks a defined enzymatic function. It runs as 56 kd and 58 kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. See U.S. Patent Publication Nos. US20080311075, US20080044379, US20080050336, US20080044380, US26090202483 and US2009020478. HCV NS5A inhibitors having fused tricyclic moieties are disclosed in International Patent Publication Nos. WO 10/065,681, WO 10/065,668, and WO 10/065,674.

Other HCV NS5A inhibitors and their use for reducing viral load in HCV infected humans have been described in U.S. Patent Publication No. US20060276511.

### SUMMARY OF THE INVENTION

In one aspect, the present invention provides Compounds of Formula (I)

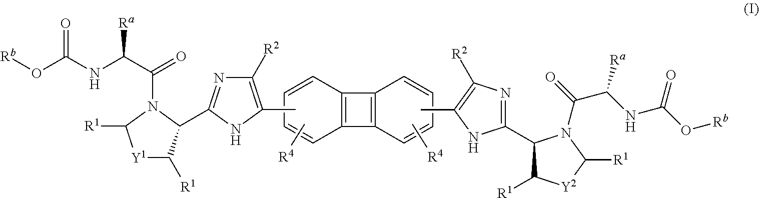

and pharmaceutically acceptable salts thereof,
wherein:

Y¹ is —C(R⁵)₂—, —CH₂C(R⁵)₂—, —OC(R⁵)₂—; or —Si(R³)₂—;

Y² is —C(R⁵)₂—, —CH₂C(R⁵)₂—, —OC(R⁵)₂—; or —Si(R³)₂—;

each occurrence of R¹ is independently selected from H, C₁-C₆ alkyl, 3- to 6-membered cycloalkyl, —CN, halo, C₁-C₆ haloalkyl, —OH, —O—(C₁-C₆ alkyl) and —O—(C₁-C₆ haloalkyl), or two R¹ groups that are attached to the same ring can optionally join to form a —(CH₂)ₘ— group, wherein said —(CH₂)ₘ— group can optionally have one or two of its —CH₂-moieties independently replaced with an N or O atom, such that when two N or O atoms are present, they are not adjacent to each other;

each occurrence of R² is independently selected from H, halo and C₁-C₆ alkyl;

each occurrence of R³ is independently selected from F, C₁-C₆ alkyl and —O—(C₁-C₆)alkyl, or two R³ groups that are attached to the same Si atom can join to form a —(CH₂)ₙ-group;

each R⁴ represents from 1 to 3 optional ring substituents, which can be the same or different, and are selected from C₁-C₆ alkyl, halo and C₁-C₆ haloalkyl;

each occurrence of R⁵ is independently selected from H, C₁-C₆ alkyl, 3- to 6-membered cycloalkyl, —CN, halo, C₁-C₆ haloalkyl, —OH, —O—(C₁-C₆ alkyl) and —O—(C₁-C₆ haloalkyl), or two R⁵ groups that are attached to the same carbon atom can optionally join to form a —(CH₂)ₙ— group, wherein said —(CH₂)ₙ— group can optionally have one or two of its —CH₂— moieties independently replaced with an N or O atom, such that when two N or O atoms are present, they are not adjacent to each other;

each occurrence, of R^{a} is independently selected from H, C₁-C₆ alkyl, phenyl, 3- to 6-membered cycloalkyl and 3- to 6-membered heterocycloalkyl, wherein said 3- to 6-membered heterocycloalkyl group contains one or two ring heteroatoms, each independently selected from N, O, S and Si.

each occurrence of R^{b} is independently selected from C₁-C₆ alkyl, 3- to 7-membered cycloalkyl and 3- to 7-membered heterocycloalkyl, wherein said 3- to 7-membered heterocycloalkyl group contains one or two ring heteroatoms, each independently selected from N, O, S and Si;

each occurrence of m is independently an integer ranging from 1 to 4; and

each occurrence of n is independently an integer ranging from 2 to 5.

The Compounds of Formula (I) (also referred to herein as the “Substituted Biphenylene Compounds”) and pharmaceutically acceptable salts thereof can be useful, for example, for inhibiting HCV viral replication or replicon activity, and for treating or preventing HCV infection in a patient. Without being bound by any specific theory, it is believed that the Substituted Biphenylene Compounds inhibit HCV viral replication by inhibiting HCV NS5A.

Accordingly, the present invention provides methods for treating or preventing HCV infection in a patient, comprising administering to the patient an effective amount of at least one Substituted Biphenylene Compound.
(a) A pharmaceutical composition comprising an effective amount of a Compound of Formula (I) or a pharmaceutically acceptable salt thereof, and a pharmaceutically acceptable carrier.
(b) The pharmaceutical composition of (a), further comprising a second therapeutic agent selected from the group consisting of HCV antiviral agents, immunomodulators, and anti-infective agents.
(c) The pharmaceutical composition of (b), wherein the HCV antiviral agent is an antiviral selected from the group consisting of HCV protease inhibitors, HCV NS5B polymerase inhibitors and HCV NS5A inhibitors.
(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.
(e) The combination of (d), wherein the HCV antiviral agent is an antiviral selected from the group consisting of HCV protease inhibitors, HCV NS5B polymerase inhibitors, and HCV NS5A inhibitors.
(f) A method of inhibiting HCV replication in a subject in need thereof which comprises administering to the subject an effective amount of a Compound of Formula (I).
(g) A method of treating HCV infection and/or reducing the likelihood or severity of symptoms of HCV infection in a subject in need thereof which comprises administering to the subject an effective amount of a Compound of Formula (I).
(h) The method of (g), wherein the Compound of Formula (I) is administered in combination with an effective amount of at least one second therapeutic agent selected from the group consisting of HCV antiviral agents, immunomodulators, and anti-infective agents.
(i). The method of (h), wherein the HCV antiviral agent is an antiviral selected from the group consisting of HCV protease inhibitors, HCV NS5B polymerase inhibitors and HCV NS5A inhibitors.
(j) A method of inhibiting HCV replication in a subject in need thereof which comprises administering to the subject the pharmaceutical composition of (a), (b) or (c) or the combination of (d) or (e).
(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) inhibiting HCV replication or (b) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (k) above are understood to include all embodiments of the compounds, including such embodiments as result from combinations of embodiments.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention provides Substituted Biphenylene Compounds, pharmaceutical compositions comprising at least one Substituted Biphenylene Compound, and methods of using the Substituted Biphenylene Compounds for treating or preventing a viral infection or a virus-related disorder in a patient.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Substituted Biphenylene Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloallyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-β-alkyl, alkylthio, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkenyl group is unsubstituted. The term “C₂-C₆ alkenyl” refers to an alkenyl group having from 2 to 6 carbon atoms.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkynyl group is unsubstituted. The term “C₂-C₆ alkynyl” refers to an alkynyl group having from 2 to 6 carbon atoms.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon/atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is unsubstituted. In another embodiment, an aryl group is phenyl.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

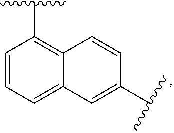 [SMILES: *c1ccc2c(*)cccc2c1]

is understood to represent both:

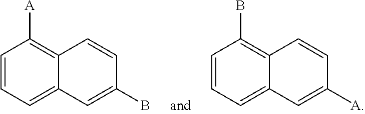

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

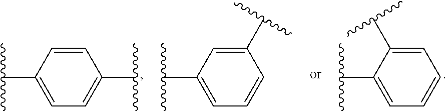

The term “cycloalkyl,” as used herein, refers to a non-aromatic mono- or multicyclic ring system Comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

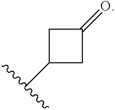 [SMILES: *C1CC(=O)C1]

The term “cycloalkenyl,” as used herein, refers to a non-aromatic mono- or multicyclic, ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is unsubstituted. In another embodiment, a cycloalkenyl group is cyclopentenyl. In another embodiment, a cycloalkenyl group is cyclohexenyl. The term “4 to 7-membered cycloalkenyl” refers to a cycloalkenyl group having from 4 to 7 ring carbon atoms.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl; imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and either available bond on a heteroarylene ring can connect to either group flanking the heteroarylene group. For example, the group “A-heteroarylene-B,” wherein the heteroarylene group is:

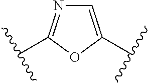 [SMILES: *c1cnc(*)o1]

is understood to represent both:

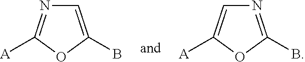

In one embodiment, a heteroarylene group is unsubstituted. In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

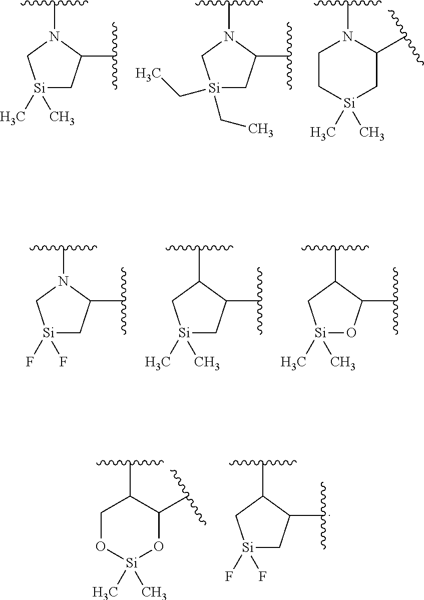

A ring carbon atom of a heterocycloalkyl group may be functionalized as a carbonyl group. An illustrative example of such a heterocycloalkyl group is:

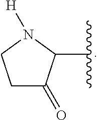 [SMILES: *C1NCCC1=O]

In one embodiment, a heterocycloalkyl group is unsubstituted. In another embodiment, a heterocycloalkyl group is a 5-membered monocyclic heterocycloalkyl. In another embodiment, a heterocycloalkyl group is a 6-membered monocyclic heterocycloalkyl. The term “3 to 7-membered monocyclic cycloalkyl” refers to a monocyclic heterocycloalkyl group having from 3 to 7 ring atoms. The term “4 to 7-membered monocyclic cycloalkyl” refers to a monocyclic heterocycloalkyl group having from 4 to 7 ring atoms. The term “7 to 11-membered bicyclic heterocycloalkyl” refers to a bicyclic heterocycloalkyl group having from 7 to 11 ring atoms.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 7 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. A ring carbon atom of a heterocycloalkenyl group may be functionalized as a carbonyl group. In one embodiment, a heterocycloalkenyl group is a 5-membered heterocycloalkenyl. In another embodiment, a heterocycloalkenyl group is a 6-membered heterocycloalkenyl. The term “4 to 7-membered heterocycloalkenyl” refers to a heterocycloalkenyl group having from 4 to 7 ring atoms. Unless otherwise indicated, a heterocycloalkenyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, and Y₁Y₂NS(O)₂—, wherein Y₁ and Y₂ can be the same or different and are independently selected from the group consisting of hydrogen, alkyl, aryl, cycloalkyl, and alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

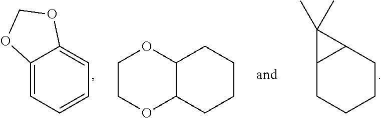

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R′, R^{a}, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Substituted Biphenylene Compound or a pharmaceutically acceptable salt or solvate of the compound. The transformation may occur by various mechanisms (e.g., by metabolic or chemical processes), such as, for example, through hydrolysis in blood.

For example, if a Substituted Biphenylene Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms; alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Substituted Biphenylene Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Substituted Biphenylene Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)allyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N— or di-N,N—(C₁-C₆)alkylaminoalkyl; —C(Y⁴)Y⁵ wherein Y⁴ is H or methyl and Y⁵ is mono-N— or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Substituted Biphenylene Compounds can form salts which are also within the scope of this invention. Reference to a Substituted Biphenylene Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Substituted Biphenylene Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Substituted Biphenylene Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Stereochemically pure compounds may also be prepared by using chiral starting materials or by employing salt resolution techniques. Also, some of the Substituted Biphenylene Compounds may be atropisomers (e.g., substituted biaryls) and are considered as part of this invention. Enantiomers can also be directly separated using chiral chromatographic techniques.

It is also possible that the Substituted Biphenylene Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Substituted Biphenylene Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Substituted Biphenylene Compounds, and of the salts, solvates, hydrates, esters and prodrugs of the Substituted Biphenylene Compounds, are intended to be included in the present invention.

The following abbreviations are used below and have the following meanings: AcCl is acetyl chloride; BOC or Boc is tert-butyloxycarbonyl; DMF is N,N-dimethylformamide; dppf is diphenylphosphinoferrocene; DMSO is dimethylsulfoxide; EtOAc is ethyl acetate; HATU is O-(7-azabenzotriazol-1-yl)-N,N,N′,N′-tetramethyluronium hexafluorophosphate; HPLC is high performance liquid chromatography; HRMS is high resolution mass spectrometry; i-Pr is isopropyl; KOH is potassium hydroxide; LCMS is liquid chromatography/mass spectrometry; LRMS is low resolution mass spectrometry; TFA is trifluoroacetic acid; THF is tetrahydrofuran; and TLC is thin-layer chromatography.

### The Compounds of Formula (I)

The present invention provides Substituted Biphenylene Compounds of Formula (I):

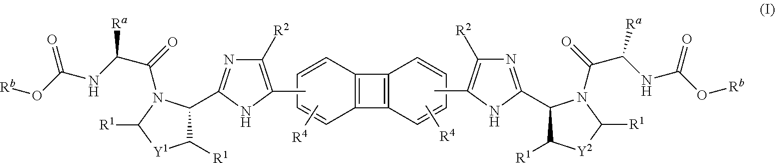

and pharmaceutically acceptable salts thereof, wherein Y¹, Y², R¹, R², R⁴, R^{a} and R^{b} are defined above for the Compounds of Formula (I).

In one embodiment, each occurrence of R¹ is H.

In one embodiment, each occurrence of R² is independently H or F.

In another embodiment, each occurrence of R² is H.

In one embodiment, each occurrence of R⁴ is H.

In another embodiment, each occurrence of R² is H or F and each occurrence of R⁴ is H.

In another embodiment, each occurrence of R² and R⁴ is H.

In one embodiment, Y¹ is —C(R⁵)₂—, —CH₂C(R⁵)₂— or —Si(R³)₂—.

In another embodiment, Y¹ is —C(R⁵)₂—.

In another embodiment, Y¹ is —CH₂C(R⁵)₂—.

In still another embodiment, Y¹ is —Si(R³)₂—.

In one embodiment, Y² is —C(R⁵)₂—, —CH₂C(R⁵)₂— or —Si(R³)₂—.

In another embodiment, Y² is —C(R⁵)₂—.

In another embodiment, Y² is —CH₂C(R⁵)₂—.

In still another embodiment, Y² is —Si(R³)₂—.

In a further embodiment, Y¹ and Y² are each independently selected from —C(R⁵)₂—, —CH₂C(R⁵)₂— and —Si(R³)₂—.

In one embodiment, Y¹ is selected from —CH₂—, —CH₂CH₂—, —C(CH₃)₂—, —CH(F)—, —CF₂—, —Si(F)₂—, —Si(CH₃)₂— and

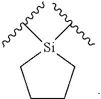 [SMILES: *[Si]1(*)CCCC1]

In another embodiment, Y² is selected from —CH₂—, —CH₂CH₂—, —C(CH₃)₂—, —CH(F)—, —CF₂—, —Si(F)₂—, —Si(CH₃)₂— and

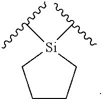 [SMILES: *[Si]1(*)CCCC1]

In another embodiment, Y¹ and Y² are each independently selected from —CH₂—, —CH₂CH₂—, —C(CH₃)₂—, —CH(F)—, —CF₂—, —Si(F)₂—, —Si(CH₃)₂— and

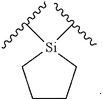 [SMILES: *[Si]1(*)CCCC1]

In one embodiment, each occurrence of R^{a} is independently C₁-C₆ alkyl.

In another embodiment, each occurrence of R^{a} is isopropyl.

In one embodiment, each occurrence of R^{b} is independently C₁-C₆ alkyl.

In another embodiment, each occurrence of R^{b} is methyl.

In one embodiment, each occurrence of R^{a} and R^{b} is independently C₁-C₆ alkyl.

In another embodiment, each occurrence of R^{a} is isopropyl and each occurrence of R^{b} is methyl.

In one embodiment, each occurrence of the two groups of formula (I) having the structures:

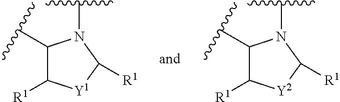

are each independently selected from:

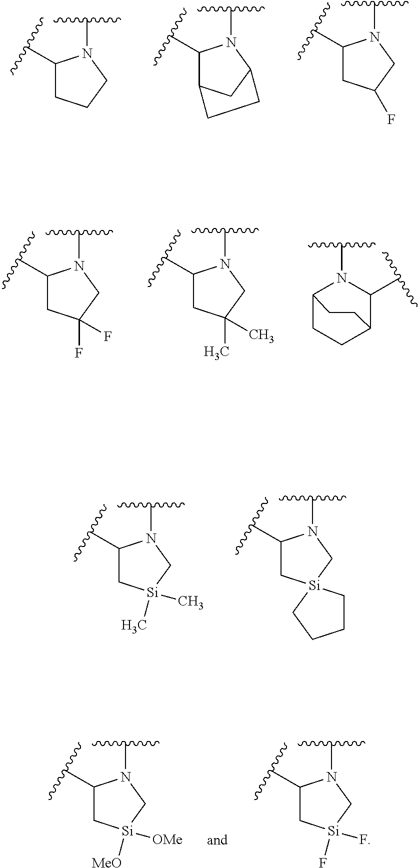

In another embodiment, each occurrence of the two groups of formula (I) having the structures:

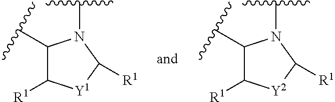

are both the same and are selected from:

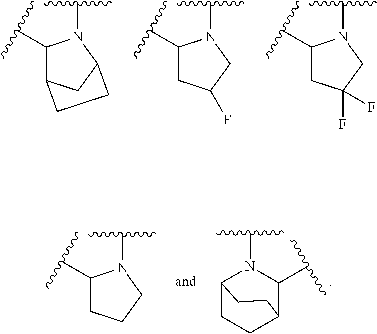 [SMILES: *C1C2CCC(C2)N1* ; *C1CC(F)CN1* ; *C1CC(F)(F)CN1* ; *C1CCCN1* ; *C1C2CCC(CC2)N1*]

In one embodiment, each occurrence of the two groups of formula (I) having the structures:

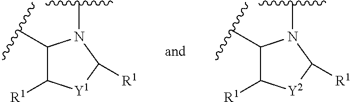

are each independently selected from:

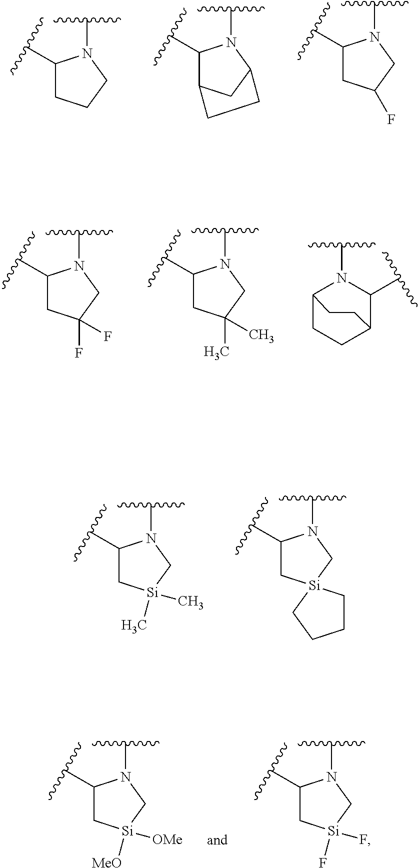

and each occurrence of R^{a} and R^{b} is independently C₁-C₆ alkyl.

In another embodiment, each occurrence of the two groups of formula (I) having the structures:

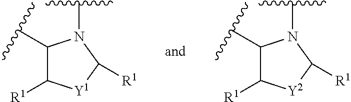

are both the same and are selected from:

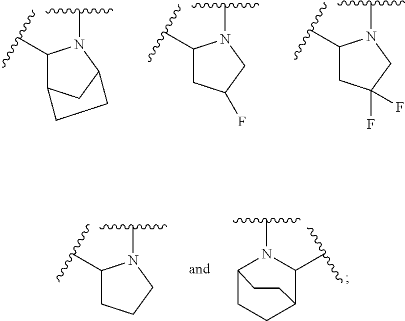 [SMILES: *C1C2CCC(C2)N1* ; *C1CC(F)CN1* ; *C1CC(F)(F)CN1* ; *C1CCCN1* ; *C1C2CCC(CC2)N1*]

each occurrence of R^{a} is isopropyl; and each occurrence of R^{b} is methyl.

In one embodiment, each occurrence of the two groups of formula (I) having the structures:

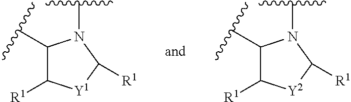

are each independently selected from:

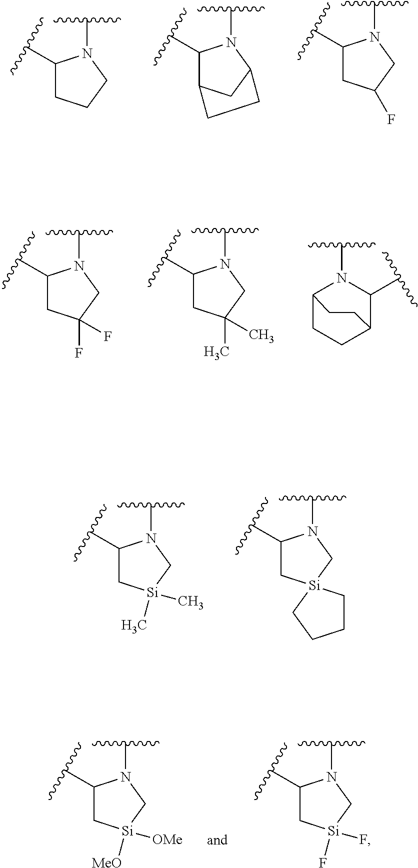

each occurrence of R^{a} and R^{b} is independently C₁-C₆ alkyl; and each occurrence of R⁴ is H.

In one embodiment, variables Y¹, Y², R¹, R², R⁴, R^{a} and R^{b} in the Compounds of Formula (I) are selected independently from each other.

In another embodiment, a Compound of Formula (I) is in substantially purified form.

In one embodiment, the Compounds of Formula (I) have the structure:

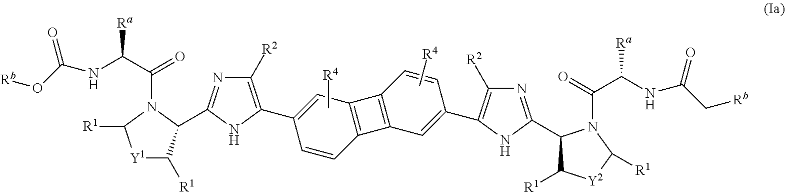

and pharmaceutically acceptable salts thereof, wherein Y¹, Y², R¹, R², R⁴, R^{a} and R^{b} are defined above for the Compounds of Formula (I).

In one embodiment, for the Compounds of Formula (Ia), each occurrence of R¹ is H.

In one embodiment, for the Compounds of Formula (Ia), each occurrence of R² is independently H or F.

In another embodiment, for the Compounds of Formula (Ia), each occurrence of R² is H.

In one embodiment, for the Compounds of Formula (Ia), each occurrence of R⁴ is H.

In another embodiment, for the Compounds of Formula (Ia), each occurrence of R² is H or F and each occurrence of R⁴ is H.

In another embodiment, for the Compounds of Formula (Ia), each occurrence of R² and R⁴ is H.

In one embodiment, for the Compounds of Formula (Ia), Y¹ is —C(R⁵)₂—, —CH₂C(R⁵)₂— or —Si(R³)₂—.

In another embodiment, for the Compounds of Formula (Ia), Y¹ is —C(R⁵)₂—.

In another embodiment, for the Compounds of Formula (Ia), Y¹ is —CH₂C(R⁵)₂—.

In still another embodiment, for the Compounds of Formula (Ia), Y¹ is —Si(R³)₂—.

In one embodiment, for the Compounds of Formula (Ia), Y² is —C(R⁵)₂—, —CH₂C(R⁵)₂— or —Si(R³)₂—.

In another embodiment, for the Compounds of Formula (Ia), Y² is —C(R⁵)₂—.

In another embodiment, for the Compounds of Formula (Ia), Y² is —CH₂C(R⁵)₂—.

In still another embodiment, for the Compounds of Formula (Ia), Y² is —Si(R³)₂—.

In a further embodiment, for the Compounds of Formula (Ia), Y¹ and Y² are each independently selected from —C(R⁵)₂—, —CH₂C(R⁵)₂— and —Si(R³)₂—.

In one embodiment, for the Compounds of Formula (Ia), Y¹ is selected from —CH₂—, —CH₂CH₂—, —C(CH₃)₂—, —CH(F)—, —CF₂—, —Si(F)₂—, —Si(CH₃)₂— and

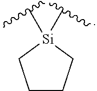 [SMILES: *[Si]1(*)CCCC1]

In another embodiment, for the Compounds of Formula (Ia), Y² is selected from —C₁₋₁₂—, —CH₂CH₂—, —C(CH₃)₂—, —CH(F)—, —CF₂—, —Si(F)₂—, —Si(CH₃)₂— and

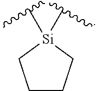 [SMILES: *[Si]1(*)CCCC1]

In another embodiment, for the Compounds of Formula (Ia), Y¹ and Y² are each independently selected from

### —CH₂—, —CH₂CH₂—, —C(CH₃)₂—, —CH(F)—, —CF₂—, —Si(F)₂—, —Si(CH₃)₂— and

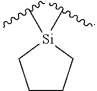 [SMILES: *[Si]1(*)CCCC1]

In one embodiment, for the Compounds of Formula (Ia), each occurrence of R³ is independently C₁-C₆ alkyl.

In another embodiment, for the Compounds of Formula (Ia), each occurrence of R^{a} is isopropyl.

In one embodiment, for the Compounds of Formula (Ia), each occurrence of R^{b} is independently C₁-C₆ alkyl.

In another embodiment, for the Compounds of Formula (Ia), each occurrence of R^{b} is methyl.

In one embodiment, for the Compounds of Formula (Ia), each occurrence of R^{a} and R^{b} is independently C₁-C₆ alkyl.

In another embodiment, for the Compounds of Formula (Ia), each occurrence of R^{a} is isopropyl and each occurrence of R^{b} is methyl.

In one embodiment, for the Compounds of Formula (Ia), each occurrence of the two groups of formula (I) having the structures:

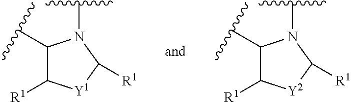

are each independently selected from:

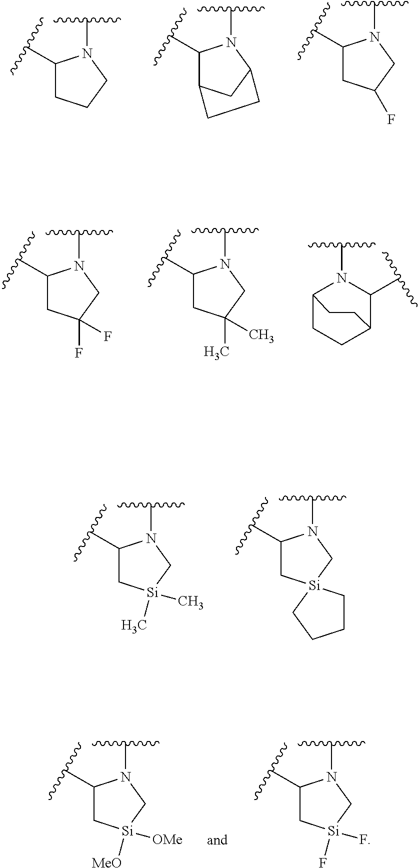

In another embodiment, for the Compounds of Formula (Ia), each occurrence of the two groups of formula (I) having the structures:

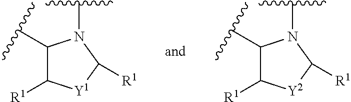

are both the same and are selected from:

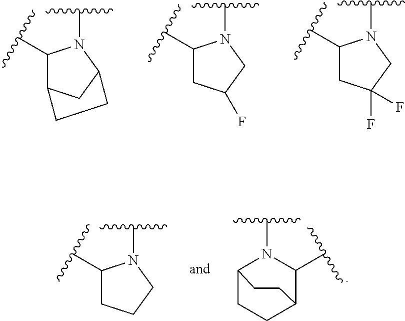 [SMILES: *C1C2CCC(C2)N1* ; *C1CC(F)CN1* ; *C1CC(F)(F)CN1* ; *C1CCCN1* ; *C1C2CCC(CC2)N1*]

In one embodiment, for the Compounds of Formula (Ia), each occurrence of the two groups of formula (I) having the structures:

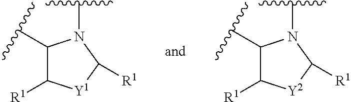

are each independently selected from:

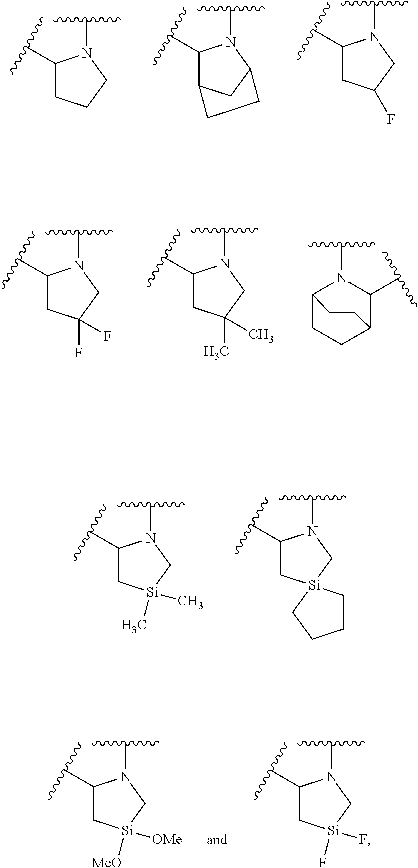

and each occurrence of R¹ and R^{b} is independently C₁-C₆ alkyl.

In another embodiment, for the Compounds of Formula (Ia), each occurrence of the two groups of formula (I) having the structures:

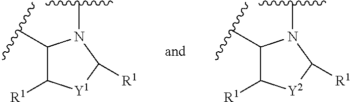

are both the same and are selected from:

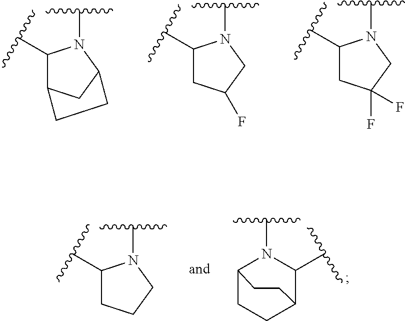 [SMILES: *C1C2CCC(C2)N1* ; *C1CC(F)CN1* ; *C1CC(F)(F)CN1* ; *C1CCCN1* ; *C1C2CCC(CC2)N1*]

each occurrence of R^{a} is isopropyl; and each occurrence of R^{b} is methyl.

In one embodiment, for the Compounds of Formula (Ia), each occurrence of the two groups of formula (I) having the structures:

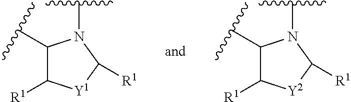

are each independently selected from:

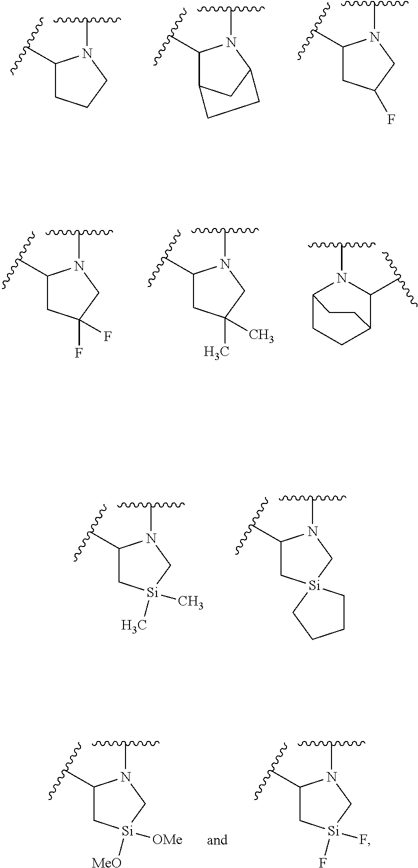

each occurrence of R^{a} and R^{b} is independently C₁-C₆ alkyl; and each occurrence of R⁴ is H.

In one embodiment, for the Compounds of Formula (Ia), for the Compounds of Formula (Ia), each occurrence of R² and R⁴ is H; each occurrence of R⁸ is, isopropyl; each occurrence of R^{b} is methyl; and the two groups of formula (Ia) having the structures:

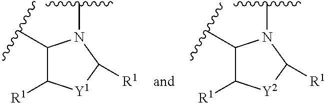

are both the same and are selected from:

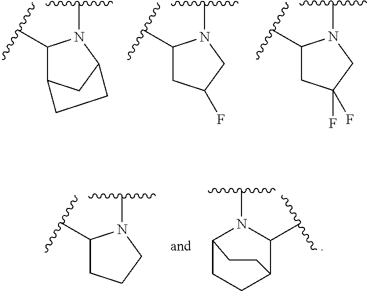 [SMILES: *C1C2CCC(C2)N1* ; *C1CC(F)CN1* ; *C1CC(F)(F)CN1* ; *C1CCCN1* ; *C1C2CCC(CC2)N1*]

In one embodiment, variables Y¹, Y², R¹, R², R⁴, R^{a} and R^{b} in the Compounds of Formula (Ia) are selected independently from each other.

In another embodiment, a Compound of Formula (Ia) is in substantially purified form:

Non-limiting examples of the Compounds of Formula (I) include compounds 1-8, as listed in the table below. Compounds 1-5 were prepared using the methods, or procedures similar to those described in the Examples section. Compounds 6-8 can be prepared using procedures that are similar to those described in the Examples section.

| Compound | |
|---|---|
| No. | Structure |
| 1 | 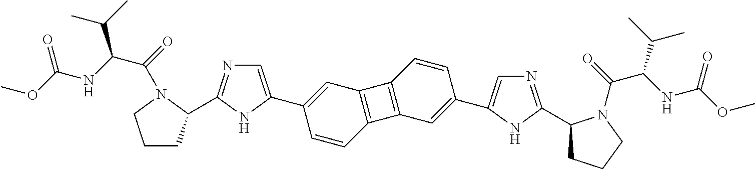 |
| 2 | 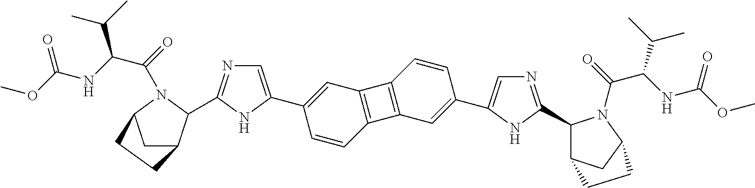 |
| 3 | 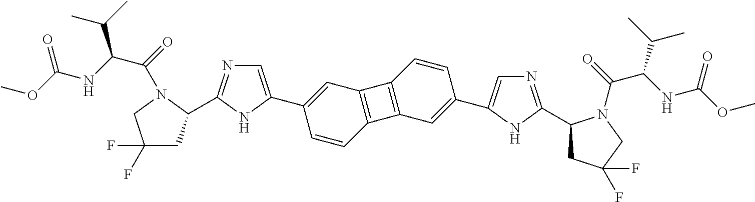 |
| 4 | 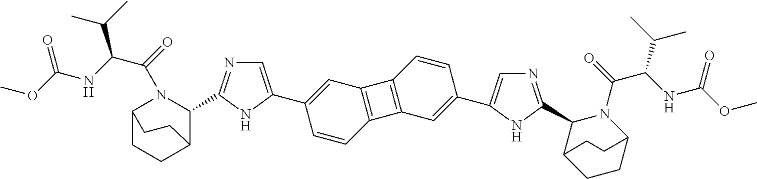 |
| 5 | 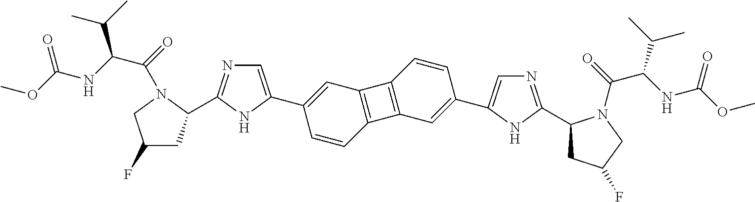 |
| 6 | 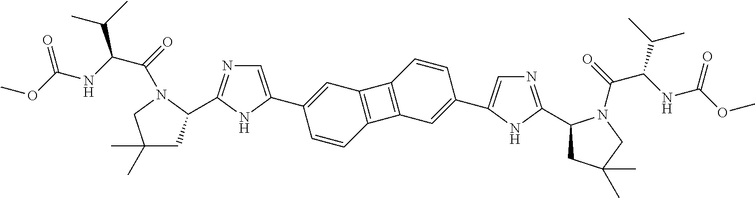 |
| 7 | 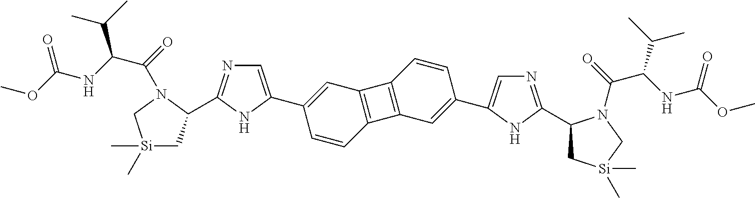 |
| 8 | 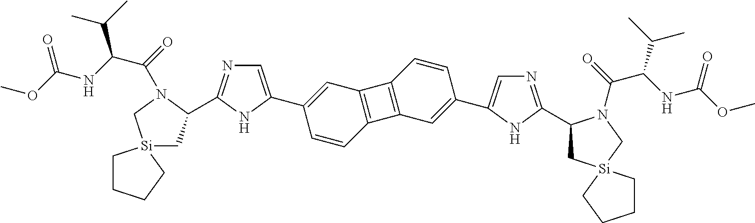 |

and pharmaceutically acceptable salts thereof.

### Methods For Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Scheme 1 below. Alternative synthetic pathways and analogous structures will be apparent to those skilled in the art of organic synthesis. All stereoisomers and tautomeric forms of the compounds are contemplated.

Some commercially available starting materials and intermediates used for the synthesis of the Compounds of Formula (I) are available which contain intact fused tricyclic ring systems. These starting materials and intermediates are available from commercial suppliers such as Sigma-Aldrich (St. Louis, Mo.) and Acros Organics Co. (Fair Lawn, N.J.). Such starting materials and intermediates compounds are used as received.

Scheme 1 shows a general method useful for making the Compounds of Formula (I).

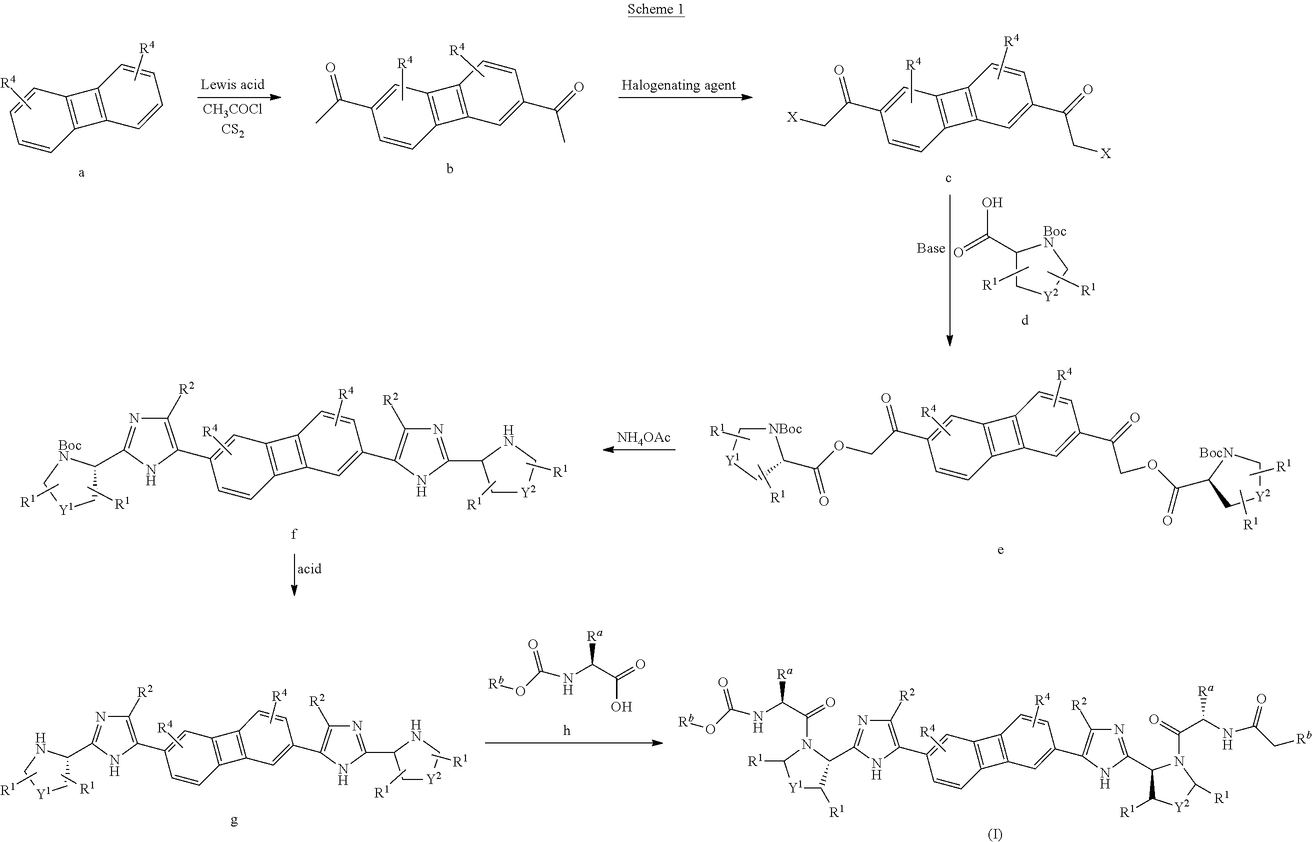

wherein X is Br, I or Cl, and Y¹, Y², R¹, R², R⁴, R^{a} and R^{b} are defined above for the Compounds of Formula (I).

Acetylation of biphenylene (a) using standard Friedel-Crafts acylation methodology provides the bis-acylated biphenylene derivative of formula b. Halogenation of the acyl groups of b using, for example, phenyltrimethylammoniumbromide, provides the bis-halo compounds of formula c. Coupling of each of the halo groups of c with a Boc-protected heterocycle of formula d in the presence of a non-nucleophilic base provides the bis keto-ester compounds of formula e. Cyclization of the keto-ester groups of the compounds of formula e in the presence of ammonium acetate provides the bis-imidazole compounds of formula f. The Boc groups off can then be removed in the presence of an acid, such as TFA, to provide the compounds of formula g. Coupling of the terminal cyclic amino groups of g with an amino acid derivative of formula h using standard amide coupling methodology provides the Compounds of Formula (I).

In some of the Substituted Biphenylene Compounds contemplated in Scheme 1, amino acids (such as, but not limited to proline, 4,4-difluoroproline, (S)-2-piperidine carboxylic acid, valine, alanine, norvaline, etc.) are incorporated as part of structures. Methods have been described in the general literature as well as in Banchard US 2009/0068140 (Published Mar. 9, 2009) for the preparation of such amino acid-derived intermediates.

One skilled in the art of organic synthesis will recognize that the synthesis of fused tricyclic cores in Formula (I) may require protection of certain functional groups (i.e., derivatization for the purpose of chemical compatibility with a particular reaction condition). Suitable protecting groups for the various functional groups of these compounds and methods for their installation and removal can be found in Greene et al., Protective Groups in Organic Synthesis, Wiley-Interscience, New York, (1999).

One skilled in the art of organic synthesis will further recognize that the synthesis of the Compounds of Formula (I) require the construction of an amide bond. Methods useful for making such amide bonds, include but are not limited to, the use of a reactive carboxy derivative (e.g., an acid halide, or ester at elevated temperatures) or the use of an acid with a coupling reagent (e.g, HOBt, EDCI, DCC, HATU, PyBrop) with an amine.

One skilled in the art of organic synthesis will also recognize that one route for the synthesis of fused bi-aryl tricyclic cores in Formula (I) may be more desirable depending on the choice of appendage substituents. Additionally, one skilled in the art will recognize that in some cases the order of reactions may differ from that presented herein to avoid functional group incompatibilities and can amend the synthetic route accordingly.

The starting materials used and the intermediates prepared using the methods set forth in the Scheme above and in the Examples below may be isolated and purified if desired using conventional techniques, including but not limited to filtration, distillation, crystallization, chromatography and alike. Such materials can be characterized using conventional means, including physical constants and spectral data.

### EXAMPLES

### General Methods

Solvents, reagents, and intermediates that are commercially available were used as received. Reagents and intermediates that are not commercially available were prepared in the manner as described below. ¹H NMR spectra were obtained on a Bruker Avance 500 (500 MHz) and are reported as ppm downfield from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min-10% CH₃CN, 5 min-95% CH₃CN, 5-7 min-95% CH₃CN, 7 min-stop. The retention time and observed parent ion are given. Flash column chromatography was performed using pre-packed normal phase silica from Biotage, Inc. or bulk silica from Fisher Scientific. Unless otherwise indicated, column chromatography was performed on flash grade silica gel using a gradient elution of hexanes/ethyl acetate, from 100% hexanes to 100% ethyl acetate.

### Example 1

### Preparation of Intermediate Compound Int-1b

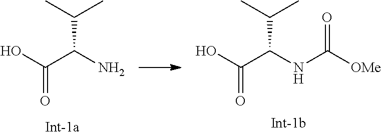

To a solution of L-valine (Int-1a, 10.0 g, 85.3 mmol) in 1M aqueous NaOH solution (86 mL) at room temperature was added solid sodium carbonate (4.60 g, 43.4 mmol). The reaction mixture was cooled to 0° C. (ice bath) and then methyl chloroformate (7.20 mL, 93.6 mmol) was added dropwise over 20 minutes. The reaction mixture was then allowed to warm to room temperature, and allowed to stir at room temperature for an additional 4 hours. The reaction mixture was then diluted with diethyl ether (100 mL), the resulting solution was cooled to at 0° C., and then concentrated hydrochloric acid (18 mL, 216 mmol) was added slowly. The reaction was extracted with EtOAc (3×100 mL) and the combined organics were dried over MgSO₄, filtered and concentrated in vacuo to provide Compound Int-1b (13.5 g, 90%), which was used without further purification.

### Example 2

### Preparation of Intermediate Compound Int-2f

### Step A—Synthesis of Compound Int-2b

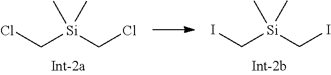

Bis(chloromethyl)dimethylsilane (Int-2a, 50 g, 0.32 mol), sodium iodide (181 g, 1.21 mol), and dried acetone (1 L) were added to a 2-liter round-bottomed flask. The resulting suspension was heated to reflux and allowed to stir at this temperature for 3.5 hours, then allowed to cool to room temperature. The reaction mixture was then filtered, concentrated in vacuo, and the residue obtained was redissolved in ethyl acetate (500 mL). The resulting suspension was filtered and the filtrate was concentrated in vacuo to provide Compound Int-2b as an oil (90.5 g, 84%), which was used without further purification.

### Step B—Synthesis of Compound Int-2d

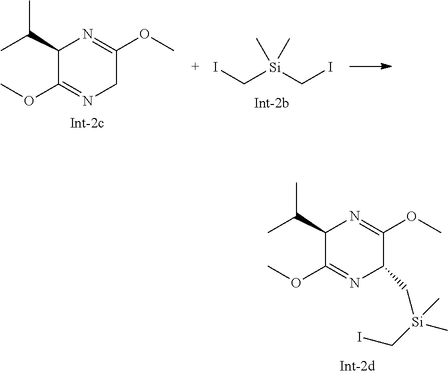

(R)-2,5-Dihydro-3,6-dimethoxy-2-isopropylpyrazine (Int-2c, 25 g, 135.7 mmol) and dry THF (500 mL) were added to a dry 1-liter flask and the resulting solution was cooled to −78° C. under a nitrogen atmosphere. A solution of 2.5 M n-BuLi in hexane (54 mL, 135 mmol) was added slowly via syringe and the resulting reaction was allowed to stir for 30 minutes at −65° C. Compound Int-2b (neat, 90.5 g, 266.2 mmol) was then added via syringe and the resulting reaction was allowed to stir for 4 hours, then was allowed to warm to room temperature gradually over a period of 1 hour. Water (100 mL) and diethyl ether (1.0 L) were added to the reaction mixture and the resulting solution was washed with water (2×200 mL) and dried over sodium sulfate, then filtered and concentrated in vacuo. The resulting residue was purified using a 330 g ISCO silica colunm/Combi-Flash system with 0-1% ether in hexanes as an eluent to provide Compound Int-2d as an oil (18.5 g, 35%).

### Step C—Synthesis of Compound Int-2e

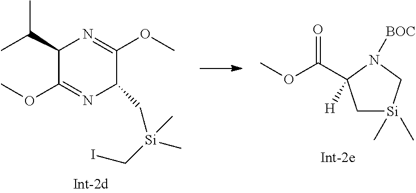

To a solution of Compound Int-2d (18.5 g, 46.7 mmol) in methanol (105 mL) was added slowly 10% aqueous HCl solution (35 mL). The resulting reaction was allowed to stir at room temperature for 5 hours and concentrated in vacuo. The residue obtained was diluted with methanol (120 mL) and concentrated in vacuo. This was repeated a total of 4 times, and the residue eventually obtained was dissolved in dichloromethane (80 mL) and diethyl ether (120 mL). To the resulting solution was added N,N-diisopropylethylamine (18 mL, 135 mmol) and the resulting reaction was allowed to stir at room temperature for 7 hours, then di-tert-butyl dicarbonate (23.5 g, 108 mmol) was added. The resulting reaction was allowed to stir at room temperature for about 15 hours, then concentrated in vacuo. The residue obtained was taken up in ethyl acetate (300 mL), washed with water (200 dried over sodium sulfate, filtered and concentrated in vacuo. The residue obtained was purified using a 330 g ISCO silica column/Combi-Flash system with 0-20% ethyl acetate in hexanes to provide Compound Int-2e as a colorless oil (8.5 g, 67%).

### Step D—Synthesis of Compound Int-2f

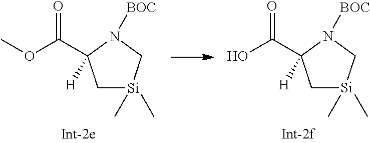

To a solution of Compound Int-2e (8.5 g, 31.1 mmol) in methanol (100 mL) was added 1.0 M aqueous KOH solution (48 mL, 48 mmol) was added. The resulting reaction was allowed to stir at room temperature for about 15 hours, then was acidified to pH˜5 using 1.0 M aqueous HCl solution (48 mL) and concentrated in vacuo. The residue obtained was extracted with dichloromethane (2×100 mL) and the combined organic extracts were concentrated in vacuo to provide Compound Int-2f as a gel (7.74 g, 96%).

### Example 3

### Intermediate Compounds Int-3a to Int-3i

The following intermediate compounds are commercially available and useful for making the Compounds of Formula (I):

| Compound | |
|---|---|
| No. | Proline Derivative |
| Int-3a | 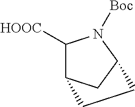 [SMILES: CC(C)(C)OC(=O)N1C(C(=O)O)[C@H]2CC[C@@H]1C2] |
| Int-3b | 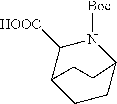 [SMILES: CC(C)(C)OC(=O)N1C2CCC(CC2)C1C(=O)O] |
| Int-3c | 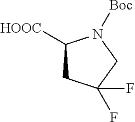 [SMILES: CC(C)(C)OC(=O)N1CC(F)(F)C[C@H]1C(=O)O] |
| Int-3d | 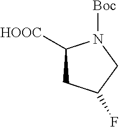 |
| Int-3e | 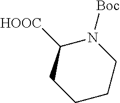 [SMILES: CC(C)(C)OC(=O)N1CCCC[C@H]1C(=O)O] |
| Int-3f | 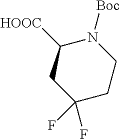 [SMILES: CC(C)(C)OC(=O)N1CCC(F)(F)C[C@H]1C(=O)O] |
| Int-3g | 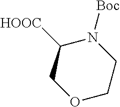 [SMILES: CC(C)(C)OC(=O)N1CCOC[C@H]1C(=O)O] |
| Int-3h | 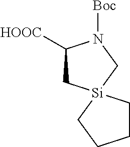 [SMILES: CC(C)(C)OC(=O)N1C[Si]2(CCCC2)C[C@H]1C(=O)O] |
| Int-3i | 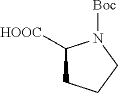 [SMILES: CC(C)(C)OC(=O)N1CCC[C@H]1C(=O)O] |

### Example 4

### Preparation of Compound 1

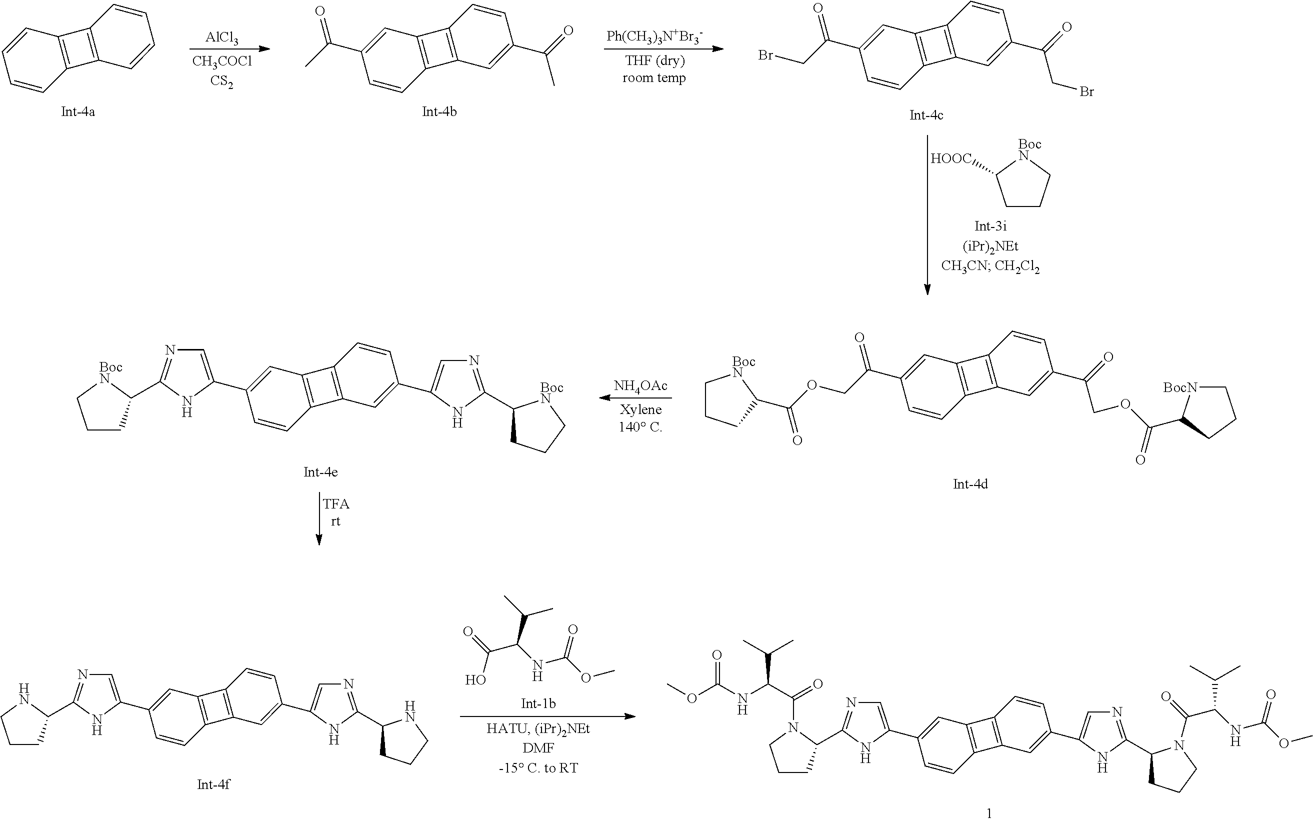

### Step A—Synthesis of Compound Int-4b

To a stirring solution of powdered aluminium chloride (3.5 g.) and carbon disulfide (10 mL) was added dropwise acetyl chloride (2 mL). The resulting reaction was allowed to stir for 0.5 hours, then biphenylene (Int-4a, 0.3 g; 0.0019 moles) was added (as a 10 mL CS₂ solution) over a 30 minute period with vigorous stirring. The resulting reaction was allowed to stir for an additional 4 hours, then the reaction mixture was poured over a mixture of ice (500 g) and 2N hydrochloric acid (100 mL). The resulting solution was extracted with EtOAc (2×100 mL). The combined organics were dried over Na₂SO₄, filtered and concentrated in vacuo and the residue obtained was purified using an ISCO silica gel flash column (100% hexanes to 20% EtOAc/hexanes) to provide Compound Int-4b (0.29 g). ¹H NMR (400 MHz, CDCl₃) δ 2.5 (s, 6H), 6.85 (d, J=64 Hz, 2H), 7.35 (d, J=6.0 Hz, 2H), 7.62 (dd, J=6 Hz, J=4H, 2H).

### Step B—Synthesis of Compound Int-4c

To an anhydrous solution containing Compound Int-4b (0.13 g, 0.00055 mol) in THF (5 mL) at room temperature was added phenyltrimethylammoniumbromide (0.42 g, 0.0011 mol) portionwise over a period of 10 minutes. The resulting reaction was stirred for 4 hours and then 50 mL of ice cold water was added and the resulting solution was allowed to stir vigorously for 1 hour. The reaction mixture was then filtered and the Compound Int-4c was collected as a solid and used in the next step without further purification.

### Step C—Synthesis of Compound Int-4d

A mixture of Compound Int-4c (0.0005 mol), Compound Int-3i (0.2 g, 1 mmol), and diisopropylethylamine (0.25°mL, 1.2 mmol) in 1:1 (CH₂Cl₂:CH₃CN) (25 mL) was allowed to stir at room temperature for 10 hours. Ethyl acetate (100 mL) was then added to the reaction mixture and the resulting solution was washed with brine (2×25 mL), dried over sodium sulfate and concentrated in vacuo. The resulting residue was purified using preparative TLC on a silica gel plate (30% ethyl acetate/hexanes) to provide Compound Int-4d (0.1 g). ¹H NMR (400 MHz, CDCl₃) [symbol] 1.5 (s, 9H), 1.55 (s, 9H), 1.9 (m, 2H), 2.1 (m, 2H), 2.32 (m, 2H), 3.42 (m, 1H), 3.5 (m, 1H), 3.6 (m, 2H), 4.42 (m, 1H), 4.5 (m, 1H), 5.1 (d, J=10 Hz, 1H), 5.28 (dd, J=10 Hz, J=5 Hz, 2H), 5.5 (d, J=10 Hz, 1H), 6.9 (m, 2H), 7.28 (s, 2H), 7.5 (m, 2H).

### Step D—Synthesis of Compound Int-4e

Compound Int-4d (0.09 g, 0.0014 mol); ammonium acetate (0.1 g, 10 eq.) and o-xylene (5 mL) were added to a 25 mL pressure vessel. The resulting reaction was heated to 140° C. and allowed to stir at this temperature for 6 hours, then cooled to room temperature and concentrated in vacuo. The residue obtained was purified using reverse phase Gilson chromatography (10% to 90% acetonitrile/water with 0.1% TFA) to provide Compound Int-4e (0.061 g). LCMS: (M+1)=623

### Step E—Synthesis of Compound Int-4f

Compound Int-4e (50 mg) was taken up in trifluoroacetic acid (2 mL) and the resulting solution was stirred at room temperature for 0.5 hours. The reaction was then concentrated in vacuo to provide Compound Int-4f as a solid (50 mg), which was used in the next step without further purification. LCMS: (M+1)=423

### Step F—Synthesis of Compound 1

A solution of Compound Int-4f (50 mg), Compound Int-1b (46 mg, 2.2 eq), Hunig's base (0.5 ml, 3.75 mmol), DMF (5 mL) was cooled to 0° C. HATU (99 mg, 2.2 eq) was added to the cooled solution and the resulting reaction was allowed to warm to room temperature with stirring over 1 hour. The reaction was quenched with 2 mL water and the resulting solution was concentrated in vacuo. The residue obtained was purified using Gilson reverse phase chromatography (0-90% acetonitrile in water with 0.1% TFA as an eluent) and the product obtained was then treated with 4N HCl/dioxane (1 mL). The resulting solution was then concentrated in vacuo to provide Compound 1 (22 mg). LCMS; Found: (M+H)⁺=737. ¹H NMR (400 MHz, CDCl₃) [symbol] 0.92 (m, 12H), 2.1 (m, 2H), 2.2 (m, 4H), 2.3 (m, 2H), 2.6 (m, 2H), 3.6 (m, 1H), 3.7 (s, 6H), 3.75 (m, 1H), 3.9 (m, 2H), 4.15 (m, 2H), 4.25 (d, J=4 Hz, 2H), 5.25 (m, 2H), 6.9 (d, J=4 Hz, 2H), 7.2 (s, 2H), 7.3 (d, J=4 Hz, 2H).

Compounds 2, 3, 4 and 5 were made using the method described above in Example 5 and substituting the appropriately substituted proline derivative for Compound Int-3i in Step C, as outlined the table below:

| Compound | Proline Derivative |
|---|---|
| No. | Used in Step C |
| 2 | 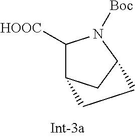 [SMILES: CC(C)(C)OC(=O)N1C(C(=O)O)[C@H]2CC[C@@H]1C2] |
| 3 | 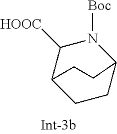 [SMILES: CC(C)(C)OC(=O)N1C2CCC(CC2)C1C(=O)O] |
| 4 | 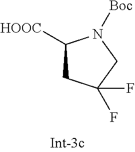 [SMILES: CC(C)(C)OC(=O)N1CC(F)(F)C[C@H]1C(=O)O] |
| 5 | 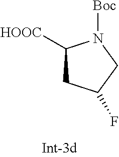 |

Data for Compound 2: LCMS; Found: (M+H)⁺=789. ¹H NMR (400 MHz, CDCl₃) δ 0.92 (m, 12H), 1.75 (m, 4H), 2.0 (m, 6H), 2.3 (m, 2H), 2.2 (m, 2H), 2.85 (m, 2H), 3.7 (s, 6H), 3.75 (m, 1H), 3.9 (m, 2H), 4.15 (m, 2H), 4.25 (d, J=4 Hz, 2H), 5.25 (m, 2H), 6.9 (d, J=4 Hz, 2H), 7.2 (s, 2H), 7.3 (d, J=4 Hz, 2H).

Data for Compound 4: LCMS; Found: (M+H)⁺=809. ¹H NMR (400 MHz, CDCl₃) 0.92 (m, 12H), 2.1 (m, 2H), 2.2 (m, 4H), 2.3 (m, 2H), 2.6 (m, 2H), 3.6 (m, 1H), 3.7 (s, 6H), 3.75 (m, 1H), 3.9 (m, 2H), 4.15 (m, 2H), 4.25 (d, J=4 Hz, 2H), 5.25 (m, 2H), 6.9 (d, J=4 Hz, 2H), 7.2 (s, 2H), 7.3 (d, J=4 Hz, 2H).

### Example 5

### Cell-Based HCV Replicon Assay

Measurement of inhibition by compounds was performed using the HCV replicon system. Several different replicons encoding different HCV genotypes or mutations were used. In addition, potency measurements were made using different formats of the replicon assay, including different ways of measurements and different plating formats. See Jan M. Vrolijk et al., A replicons-based bioassay for the measurement of interferons in patients with chronic hepatitis C, 110 J. VIROLOGICAL METHODS 201 (2003); Steven S. Carroll et al., Inhibition of Hepatitis C Virus RNA Replication by 2′-Modified Nucleoside Analogs, 278(14) J. BIOLOGICAL CHEMISTRY 11979 (2003). However, the underlying principles are common to all of these determinations, and are outlined below.

TaqMan®-Based Assay Protocol: Compounds 1, 2 and 3 were assayed for cell-based anti-HCV activity by the following protocol. Replicon cells were seeded at 5000 cells/well in 96-well collagen I-coated Nunc plates in the presence of the test compound. Various concentrations of test compound, typically in 10 serial 2-fold dilutions, were added to the assay mixture, with the starting concentration ranging from 250 μM to 1 μM. The final concentration of DMSO was 0.5%, fetal bovine serum was 5%, in the assay media. Cells were harvested on day 3 by the addition of 1× cell lysis buffer (Ambion cat #8721). The replicon RNA level was measured using real time PCR (TaqMan® assay). The amplicon was located in 5B. The PCR primers were: 5B.2F, ATGGACAGGCGCCCTGA (SEQ. ID NO. 1); 5B.2R, TTGATGGGCAGCTTGGTTTC (SEQ. ID NO. 2); the probe sequence was FAM-labeled CACGCCATGCGCTGCGG (SEQ. ID NO. 3). GAPDH RNA was used as endogenous control and was amplified in the same reaction as NS5B (multiplex PCR) using primers and VIC-labeled probe recommended by the manufacturer (PE Applied Biosystem). The real-time RT-PCR reactions were run on ABI PRISM 7900HT Sequence Detection System using the following program: 48° C. for 30 min, 95° C. for 10 min, 40 cycles of 95° C. for 15 sec, 60° C. for 1 min. The ACT values (CT_{5B}-CT_{GAPDH}) were plotted against the concentration of test compound and fitted to the sigmoid dose-response model using XLfit4 (MDL). EC₅₀, was defined as the concentration of inhibitor necessary to achieve ΔCT=1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All TaqMan® reagents were from PE Applied Biosystems. Such an assay procedure was described in detail in e.g. Malcolm et al., Antimicrobial Agents and Chemotherapy 50: 1013-1020 (2006).

The study of the HCV life cycle has been difficult due to the lack of a cell-culture system to support the HCV virus. To date, compounds in different structural classes acting on different sites within the HCV polyprotein have demonstrated efficacy in various species, including humans, in reducing HCV viral titers. Furthermore, the subgenomic replicon assay is highly correlated with efficacy in non-humans and humans infected with HCV. See K. del Carmen et al., Annals of Hepatology, 2004, 3:54.

It is accepted that the HCV replicon system described above is useful for the development and the evaluation of antiviral drugs. See Pietschmann, T. & Bartenschlager, R., Current. Opinion in Drug Discovery Research 2001, 4:657-664).

HCV replicon assay data was calculated for genotypes 1a, 1b, 2a and 3a using this method and is provided in the table below.

| | |
|---|---|
| Compound | Genotype EC₅₀ (nM) |

| No. | 1a | 1b | 2a | 3a |
|---|---|---|---|---|
| 1 | 0.03 | 0.007 | <0.019 | <0.19 |
| 2 | 0.52 | 0.005 | <0.019 | <0.19 |
| 3 | 0.05 | 0.003 | <0.019 | 0.022 |

### Uses of the Substituted Biphenylene Compounds

The Substituted Biphenylene Compounds are useful in human and veterinary medicine for treating or preventing a viral infection in a patient. In one embodiment, the Substituted Biphenylene Compounds can be inhibitors of viral replication. In another embodiment, the Substituted Biphenylene Compounds can be inhibitors of HCV replication. Accordingly, the Substituted Biphenylene Compounds are useful for treating viral infections, such as HCV. In accordance with the invention, the Substituted Biphenylene Compounds can be administered to a patient in need of treatment or prevention of a viral infection.

Accordingly, in one embodiment, the invention provides methods for treating a viral infection in a patient comprising administering to the patient an effective amount of at least one Substituted Biphenylene Compound or a pharmaceutically acceptable salt thereof.

### Treatment or Prevention of a Flaviviridae Virus

The Substituted Biphenylene Compounds can be useful for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-borne encephalitis, West Nile encephalitis, yellow fever and Hepatitis C Virus (HCV) infection.

In one embodiment, the Flaviviridae infection being treated is hepatitis C virus infection:

### Treatment or Prevention of HCV Infection

The Substituted Biphenylene Compounds are useful in the inhibition of HCV (e.g., HCV NS5A), the treatment of HCV infection and/or reduction of the likelihood or severity of symptoms of HCV infection and the inhibition of HCV viral replication and/or HCV viral production in a cell-based system. For example, the Substituted Biphenylene Compounds are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Substituted Biphenylene Compound or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The Substituted Biphenylene Compounds are also useful in the preparation and execution of screening assays for antiviral compounds. For example the Substituted Biphenylene Compounds are useful for identifying resistant HCV replicon cell lines harboring mutations within NS5A, which are excellent screening tools for more powerful antiviral compounds. Furthermore, the Substituted Biphenylene Compounds are useful in establishing or determining the binding site of other antivirals to the HCV replicase.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Combination Therapy

In another embodiment, the present methods for treating or preventing HCV infection can further comprise the administration of one or more additional therapeutic agents which are not Substituted Biphenylene Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Substituted Biphenylene Compound, or a pharmaceutically acceptable salt thereof, and (ii) at least one additional therapeutic agent that is other than a Substituted Biphenylene Compound, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Substituted Biphenylene Compound and an additional therapeutic agent may be present in fixed amounts (dosage amounts) in a single dosage unit (e.g., a capsule, a tablet and the like).

In one embodiment, the at least one Substituted Biphenylene Compound is administered during a time when the additional therapeutic agent(s) exert their prophylactic or therapeutic effect, or vice versa.

In another embodiment, the at least one Substituted Biphenylene Compound and the additional therapeutic agent(s) are administered in doses commonly employed when such agents are used as monotherapy for treating a viral infection.

In another embodiment, the at least one Substituted Biphenylene Compound and the additional therapeutic agent(s) are administered in doses lower than the doses commonly employed when such agents are used as monotherapy for treating a viral infection.

In still another embodiment, the at least one Substituted Biphenylene Compound and the additional therapeutic agent(s) act synergistically and are administered in doses lower than the doses commonly employed when such agents are used as monotherapy for treating a viral infection.

In one embodiment, the at least one Substituted Biphenylene Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Substituted Biphenylene Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Substituted Biphenylene Compound and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082,484, WO 08/082,488, WO 08/083,351, WO 08/136,815, WO 09/032,116, WO 09/032,123, WO 09/032,124 and WO 09/032,125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), IFN-α-2b-XL (Flamel Technologies), and consensus interferon as defined by determination of a consensus sequence of naturally occurring interferon alphas (Infergen™, Amgen, Thousand Oaks, Calif.).

Antibody therapy agents useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhibitors useful in the present compositions and methods include, but are not limited to, an HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124,148.

Additional HCV protease inhibitors useful in the present compositions and methods include, but are not limited to, SCH503034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), MK-7009 (Merck), TMC-435350 (Medivir), ITMN-1911R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhibitors useful in the present compositions and methods include, but are not limited to, those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Publication Nos: US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, the following compounds:

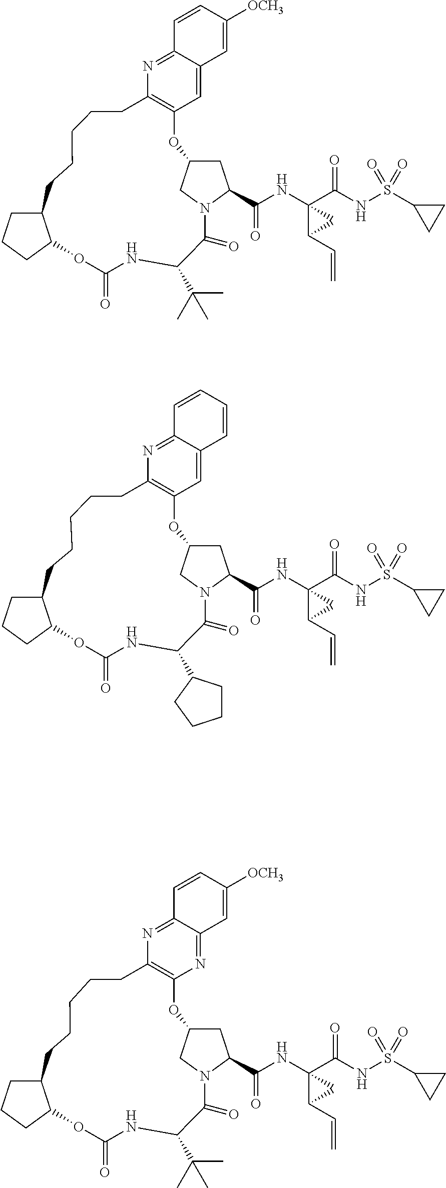

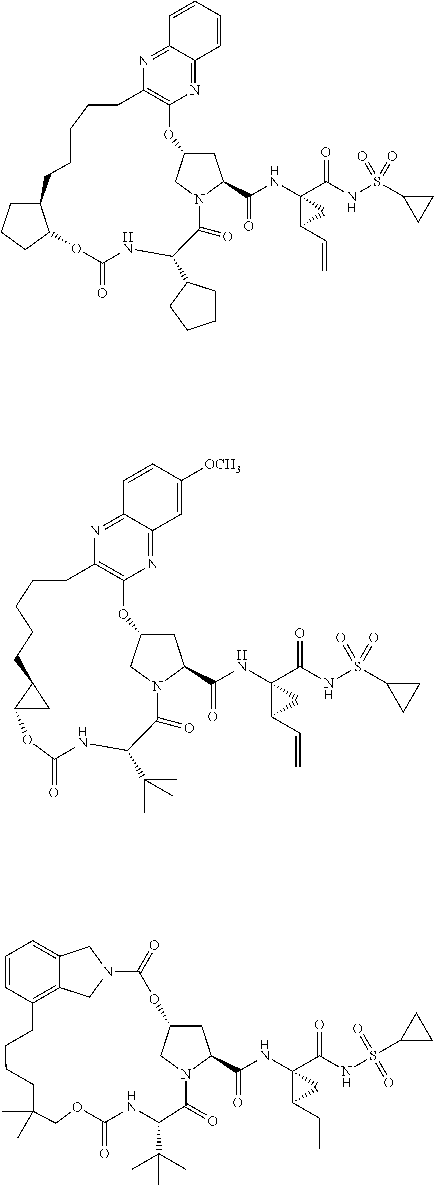

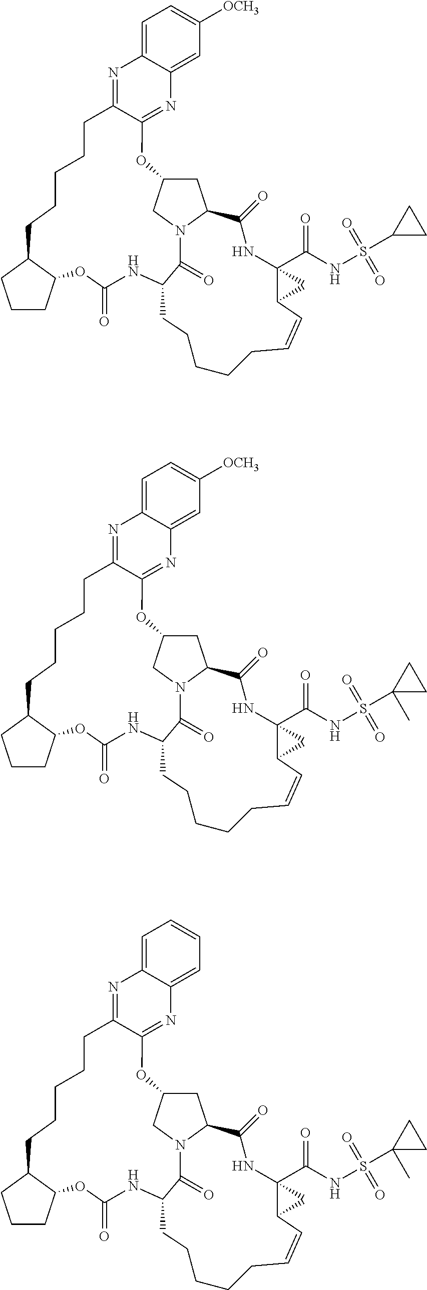 [SMILES: COc1ccc2nc3c(nc2c1)O[C@@H]1C[C@H]2C(=O)N[C@]4(C(=O)NS(=O)(=O)C5CC5)C[C@H]4/C=C\CCCCC[C@H](NC(=O)O[C@@H]4CCC[C@H]4CCCCC3)C(=O)N2C1 ; COc1ccc2nc3c(nc2c1)O[C@@H]1C[C@H]2C(=O)N[C@]4(C(=O)NS(=O)(=O)C5(C)CC5)C[C@H]4/C=C\CCCCC[C@H](NC(=O)O[C@@H]4CCC[C@H]4CCCCC3)C(=O)N2C1 ; CC1(S(=O)(=O)NC(=O)[C@@]23C[C@H]2/C=C\CCCCC[C@@H]2NC(=O)O[C@@H]4CCC[C@H]4CCCCCc4nc5ccccc5nc4O[C@@H]4C[C@@H](C(=O)N3)N(C4)C2=O)CC1]

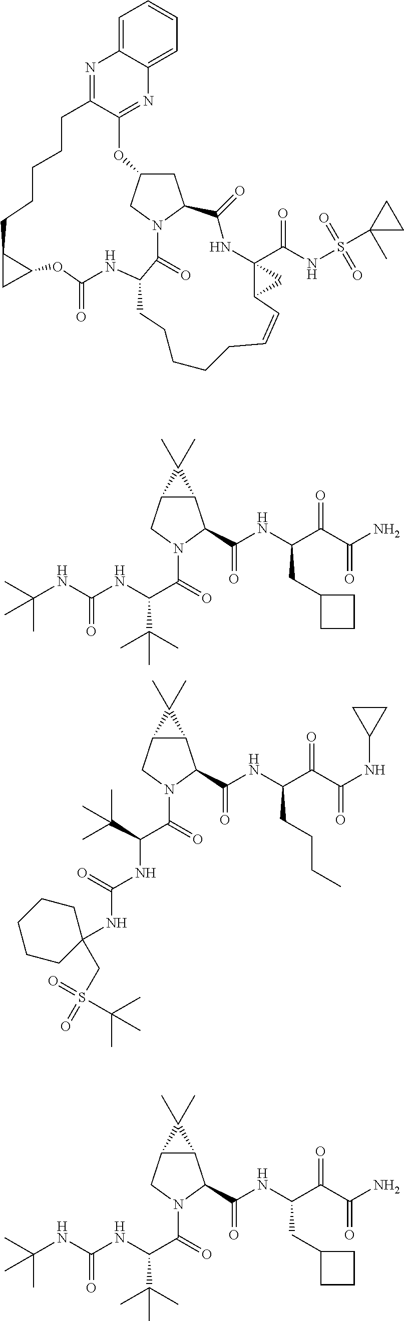

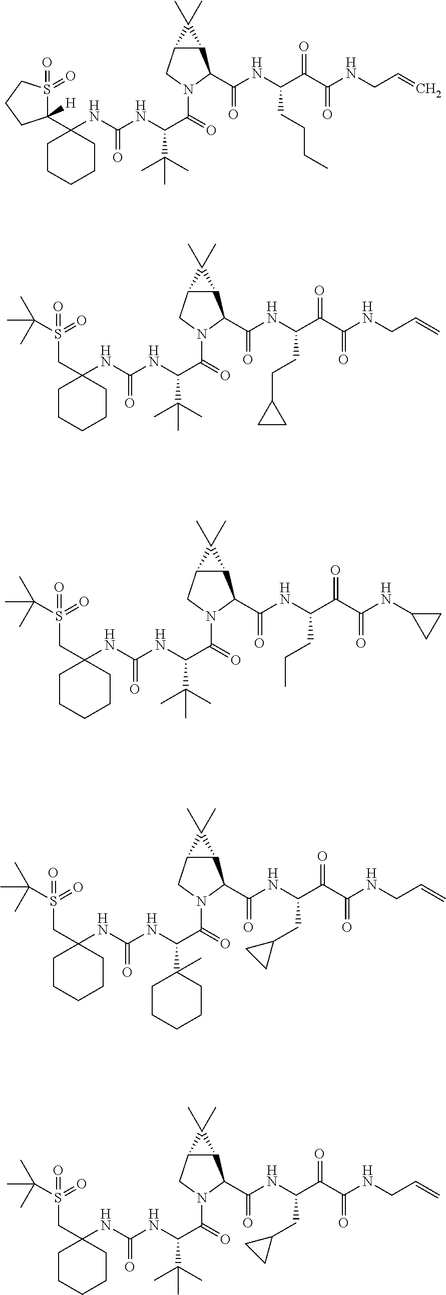

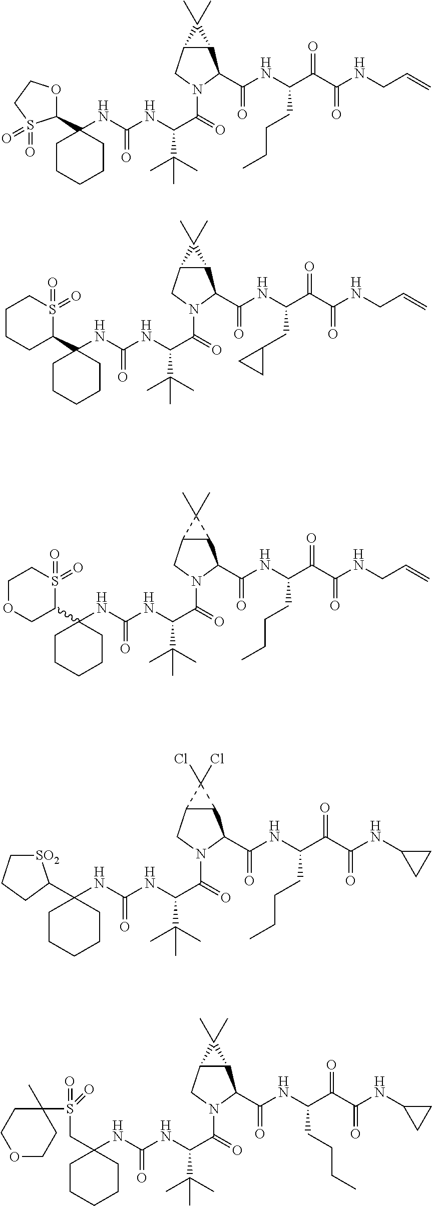

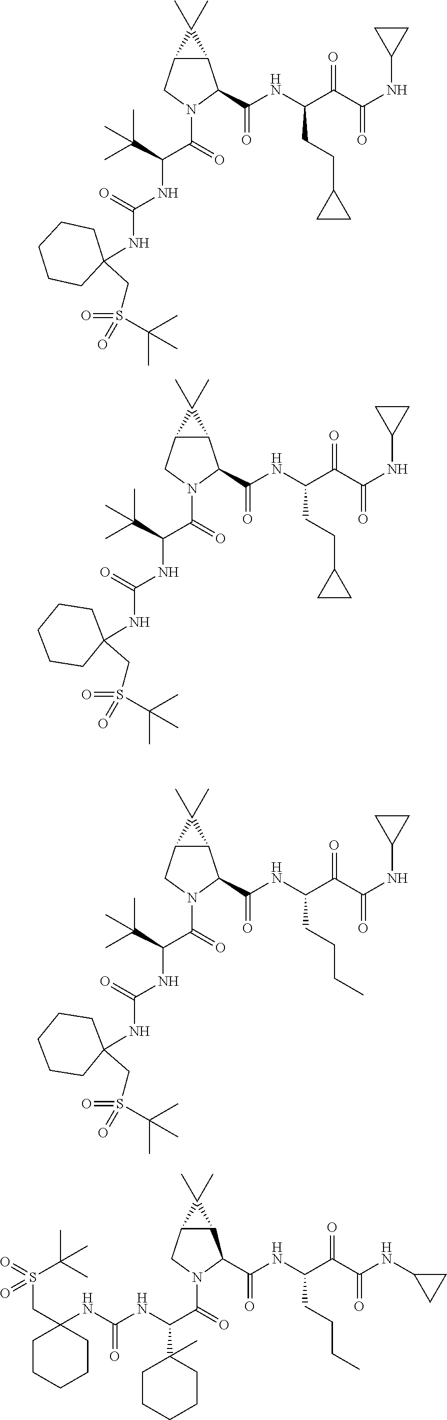

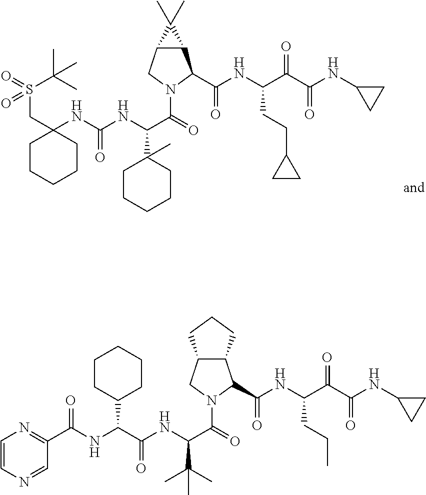

and pharmaceutically acceptable salts thereof.

Viral replication inhibitors useful in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), BMS-790052 (Bristol-Myers Squibb, see Gao et al., Nature, 465:96-100 (2010)), viramidine, A-831 (Arrow Therapeutics); an antisense agent or a therapeutic vaccine.

HCV NS4A inhibitors useful in the useful in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful in the useful in the present compositions and methods include, but are not limited to, AZD2836 (Astra Zeneca) and ACH-806 (Achillon Pharmaceuticals, New Haven, Conn.).

HCV replicase inhibitors useful in the useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C (Inovio/Tripep), PeviPRO™ (Pevion Biotect), HCV/MF59 (Chiron/Novartis) and Civacir (NABI).

Examples of further additional therapeutic agents useful in the present compositions and methods include, but are not limited to, Ritonavir (Abbott), TT033 (Benitec/Tacere Bio/Pfizer), Sima-034 (Sima Therapeutics), GNI-104 (GENimmune), GI-5005 (GlobeImmune), IDX-102 (Idenix), Levovirin™ (ICN Pharmaceuticals, Costa Mesa, Calif.); Humax (Genmab), 1TX-2155 (Ithrex/Novartis), PRO 206 (Progenies), HepaCide-I (NanoVirocides), MX3235 (Migenix), SCY-635 (Scynexis); KPE02003002 (Kemin Pharma), Lenocta (VioQuest Pharmaceuticals), LET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the Substituted Biphenylene Compounds) and the other agent(s) can be administered simultaneously (i.e., in the same composition or in separate compositions one right after the other) or sequentially. This particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

Generally, a total daily dosage of the at least one Substituted Biphenylene Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses.

In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the additional therapeutic agent is INTRON-A interferon alpha 2b (commercially available from Schering-Plough Corp.), this agent is administered by subcutaneous injection at 3MIU(12 mcg)/0.5 mL/TIW for 24 weeks or 48 weeks for first time treatment.

In another embodiment, when the additional therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the additional therapeutic agent is ROFERON A interferon alpha 2a (commercially available from Hoffmann-La Roche), this agent is administered by subcutaneous or intramuscular injection at 3MIU(11.1 mcg/mL)/TIW for at least 48 to 52 weeks, or alternatively 6MIU/TIW for 12 weeks followed by 3MIU/TIW for 36 weeks.

In still another embodiment, when the additional therapeutic agent is PEGASUS interferon alpha 2a pegylated (commercially available from Hoffmann-La Roche), this agent is administered by subcutaneous injection at 180 mcg/1 mL or 180 mcg/0.5 mL, once a week for at least 24 weeks.

In yet another embodiment, when the additional therapeutic agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, one or more compounds of the present invention are administered with one or more additional therapeutic agents selected from an HCV protease inhibitor, an HCV polymerase inhibitor, an HCV replication inhibitor, a nucleoside, an interferon, a pegylated interferon and ribavirin. The combination therapies can include any combination of these additional therapeutic agents.

In another embodiment, one or more compounds of the present invention are administered with one additional therapeutic agent selected from an HCV protease inhibitor, an interferon, a pegylated interferon and ribavirin.

In still another embodiment, one or more compounds of the present invention are administered with two additional therapeutic agents selected from an HCV protease inhibitor, an HCV replication inhibitor, a nucleoside, an interferon, a pegylated interferon and ribavirin.

In another embodiment, one or more compounds of the present invention are administered with an HCV protease inhibitor and ribavirin. In another specific embodiment, one or more compounds of the present invention are administered with a pegylated interferon and ribavirin.

In another embodiment, one or more compounds of the present invention are administered with three additional therapeutic agents selected from an HCV protease inhibitor, an HCV replication inhibitor, a nucleoside, an interferon, a pegylated interferon and ribavirin.

In one embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and a viral replication inhibitor. In another embodiment, one or more compounds of the present invention are administered with one or more additional therapeutic agents selected from an HCV polymerase inhibitor, a viral protease inhibitor, an interferon, and ribavirin.

In one embodiment, one or more compounds of the present invention are administered with one additional therapeutic agent selected from an HCV polymerase inhibitor, a viral protease inhibitor, an interferon, and a viral replication inhibitor. In another embodiment, one or more compounds of the present invention are administered with ribavirin.

In one embodiment, one or more compounds of the present invention are administered with two additional therapeutic agents selected from an HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir.

In a further embodiment, one or more compounds of the present invention are administered with ribavirin, interferon and an HCV polymerase inhibitor.

In another embodiment, one or more compounds of the present invention are administered with pegylated-interferon alpha and ribavirin.

### Compositions and Administration

Due to their activity, the Substituted Biphenylene Compounds are useful in veterinary and human medicine. As described above, the Substituted Biphenylene Compounds are useful for treating or preventing HCV infection in a patient in need thereof.

When administered to a patient, the Substituted Biphenylene Compounds can be administered as a component of a composition that comprises a pharmaceutically acceptable carrier or vehicle. The present invention provides pharmaceutical compositions comprising an effective amount of at least one Substituted Biphenylene Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid, dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Substituted Biphenylene Compounds are administered orally.

In another embodiment, the one or more Substituted Biphenylene Compounds are administered intravenously.

In another embodiment, the one or more Substituted Biphenylene Compounds are administered topically.

In still another embodiment, the one or more Substituted Biphenylene Compounds are administered sublingually.

In one embodiment, a pharmaceutical preparation comprising at least one Substituted Biphenylene Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Substituted Biphenylene Compound(s) by weight or volume. In various embodiments, the present compositions can contain, in one embodiment, from about 1% to about 70% or from about 5% to about 60% of the Substituted Biphenylene Compound(s) by weight or volume.

The quantity of Substituted Biphenylene Compound in a unit dose of preparation may be varied or adjusted from about 1 mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Substituted Biphenylene Compounds will be regulated according to the judgment of the attending clinician considering such factors as age, condition and size of the patient as well as severity of the symptoms being treated. Generally, a total daily dosage of the Substituted Biphenylene Compounds range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses.

In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Substituted Biphenylene Compound or a pharmaceutically acceptable salt thereof; (ii) one or more additional therapeutic agents that are not a Substituted Biphenylene Compound; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and two additional therapeutic agents, each of which are independently selected from the group consisting of HCV antiviral agents, immunomodulators, and anti-infective agents.

### Kits

In one aspect, the present invention provides a kit comprising a therapeutically effective amount of at least one Substituted Biphenylene Compound, or a pharmaceutically acceptable salt, solvate, ester or prodrug of said compound and a pharmaceutically acceptable carrier, vehicle or diluent.

In another aspect the present invention provides a kit comprising an amount of at least one Substituted Biphenylene Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Substituted Biphenylene Compounds and the one or more additional therapeutic agents are provided in the same container. In one embodiment, the one or more Substituted Biphenylene Compounds and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 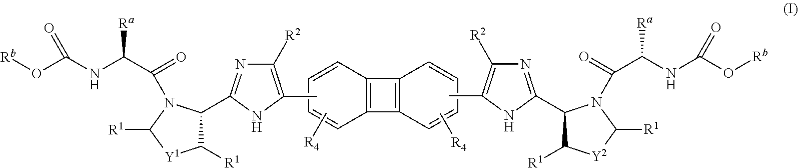
or a pharmaceutically acceptable salt thereof,
wherein:
Y¹ is —C(R⁵)₂—, —CH₂C(R⁵)₂—, —OC(R⁵)₂—; or —Si(R³)₂—;
Y² is —C(R⁵)₂—, —CH₂C(R⁵)₂—, —OC(R⁵)₂—; or —Si(R³)₂—;
each occurrence of R¹ is independently selected from H, C₁-C₆ alkyl, 3- to 6-membered cycloalkyl, —CN, halo, C₁-C₆ haloalkyl, —OH, —O—(C₁-C₆ alkyl) and —O—(C₁-C₆ haloalkyl), or two R¹ groups that are attached to the same ring can optionally join to form a —(CH₂)ₘ— group, wherein said —(CH₂)ₘ— group can optionally have one or two of its —CH₂— moieties independently replaced with an N or O atom, such that when two N or O atoms are present, they are not adjacent to each other;
each occurrence of R² is independently selected from H, halo and C₁-C₆ alkyl;
each occurrence of R³ is independently selected from F, C₁-C₆ alkyl and —O—(C₁-C₆)alkyl, or two R³ groups that are attached to the same Si atom can join to form a —(CH₂)ₙ— group;
each R⁴ represents from 1 to 3 optional ring substituents, which can be the same or different, and are selected from C₁-C₆ alkyl, halo and C₁-C₆ haloalkyl;
each occurrence of R⁵ is independently selected from H, C₁-C₆ alkyl, 3- to 6-membered cycloalkyl, —CN, halo, C₁-C₆ haloalkyl, —OH, —O—(C₁-C₆ alkyl) and —O—(C₁-C₆ haloalkyl), or two R⁵ groups that are attached to the same carbon atom can optionally join to form a —(CH₂)ₙ— group, wherein said —(CH₂)ₙ— group can optionally have one or two of its —CH₂— moieties independently replaced with an N or O atom, such that when two N or O atoms are present, they are not adjacent to each other;
each occurrence of R^{a} is independently selected from H, C₁-C₆ alkyl, phenyl, 3- to 6-membered cycloalkyl and 3- to 6-membered heterocycloalkyl, wherein said 3- to 6-membered heterocycloalkyl group contains one or two ring heteroatoms, each independently selected from N, O, S and Si.
each occurrence of R^{b} is independently selected from C₁-C₆ alkyl, 3- to 7-membered cycloalkyl and 3- to 7-membered heterocycloalkyl, wherein said 3- to 7-membered heterocycloalkyl group contains one or two ring heteroatoms, each independently selected from N, O, S and Si;
each occurrence of m is independently an integer ranging from 1 to 4; and
each occurrence of n is independently an integer ranging from 2 to 5.

2. The compound of claim 1, having the structure:

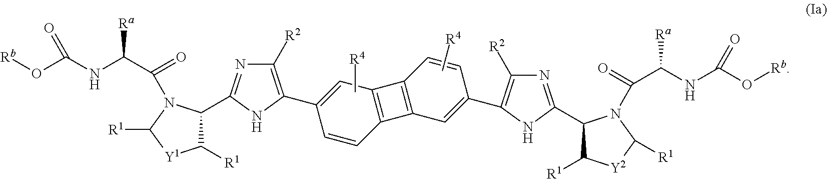

3. The compound of claim 1, wherein each occurrence of R⁴ is H.

4. The compound of claim 1, wherein each occurrence of R² is H or F.

5. The compound of claim 1, wherein each occurrence of Y² is independently selected from —CH₂—, —CH₂CH₂—, —C(CH₃)₂—, —CH(F)—, —CF₂—, —Si(F)₂—, —Si(CH₃)₂— and

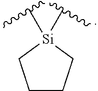 [SMILES: *[Si]1(*)CCCC1]

6. The compound of claim 5, wherein each occurrence of R^{a} is independently C₁-C₆ alkyl.

7. The compound of claim 6, wherein each occurrence of R^{b} is independently C₁-C₆ alkyl.

8. The compound of claim 1, wherein the two groups of formula (I) having the structures: 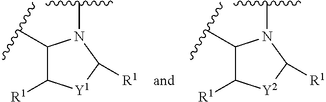
are each independently selected from:

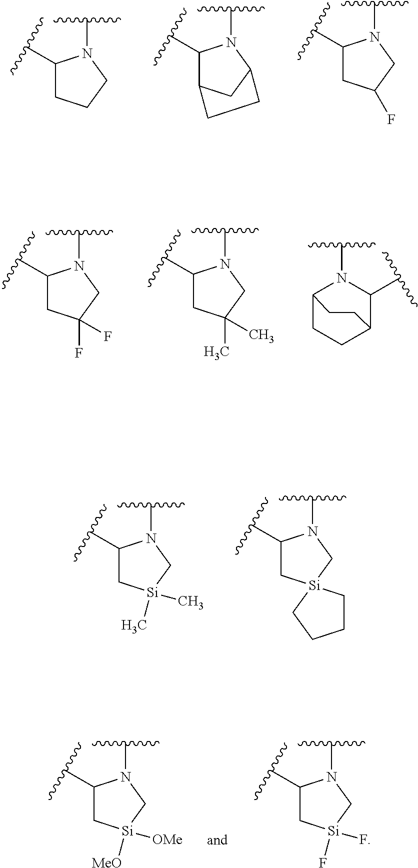

9. The compound of claim 7, wherein the two groups of formula (I) having the structures: 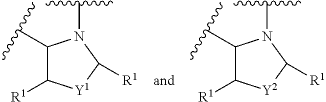
are both the same and are selected from:

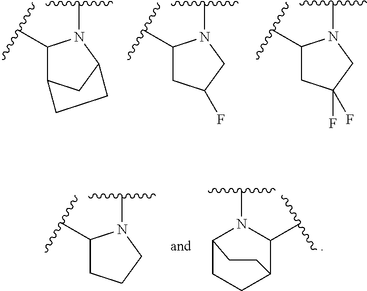 [SMILES: *C1C2CCC(C2)N1* ; *C1CC(F)CN1* ; *C1CC(F)(F)CN1* ; *C1CCCN1* ; *C1C2CCC(CC2)N1*]

10. The compound of claim 2, wherein each occurrence of R² and R⁴ is H; each occurrence of R^{a} is isopropyl; each occurrence of R^{b} is methyl; and the two groups of formula (Ia) having the structures: 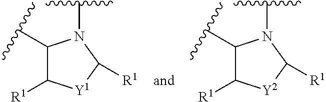
are both the same and are selected from:

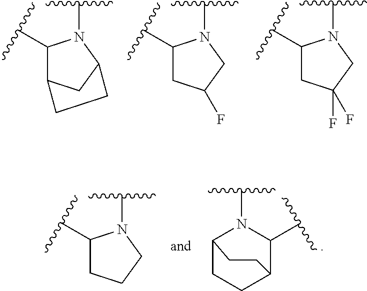 [SMILES: *C1C2CCC(C2)N1* ; *C1CC(F)CN1* ; *C1CC(F)(F)CN1* ; *C1CCCN1* ; *C1C2CCC(CC2)N1*]

11. The compound, having the structure: 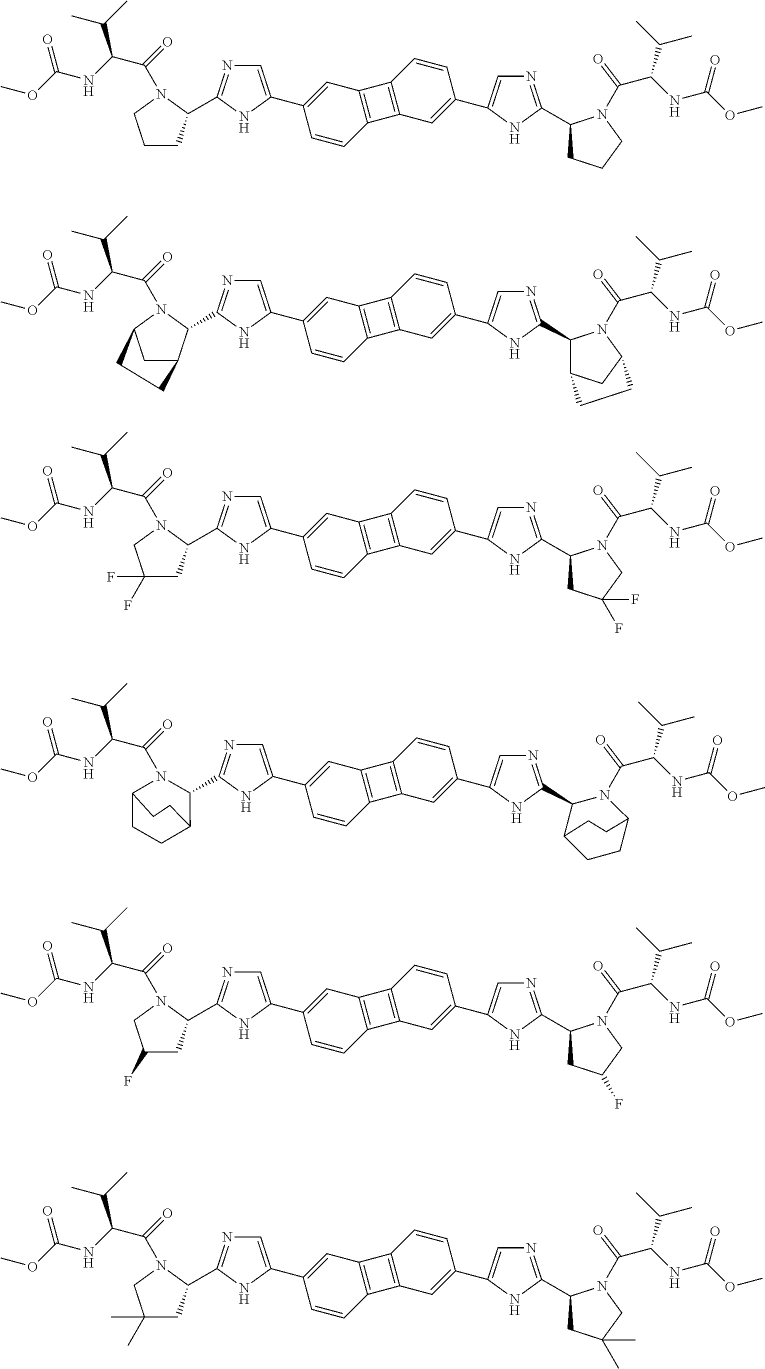 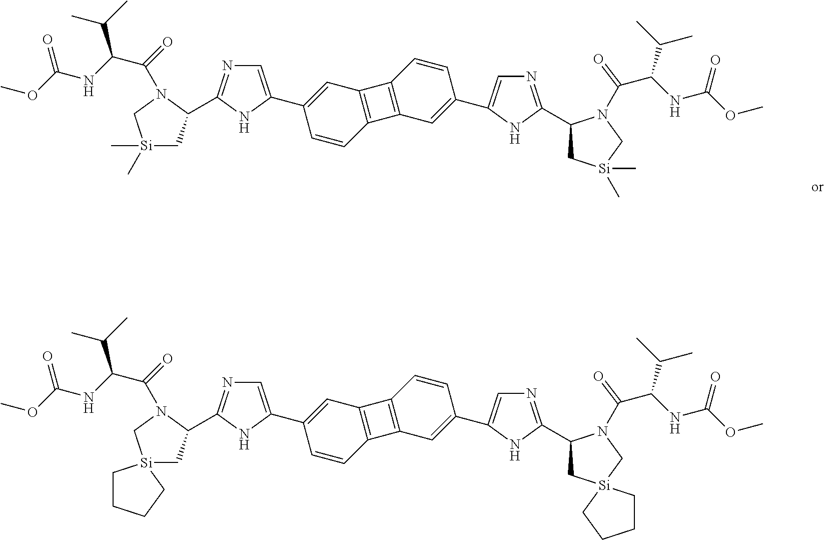
or a pharmaceutically acceptable salt thereof.

12. A pharmaceutical composition comprising an effective amount of the compound of claim 1, or a pharmaceutically acceptable salt thereof, and a pharmaceutically acceptable carrier.

13. The pharmaceutical composition according to claim 12, further comprising a second therapeutic agent selected from the group consisting of HCV antiviral agents, immunomodulators, and anti-infective agents.

14. The pharmaceutical composition according to claim 13, further comprising a third therapeutic agent selected from the group consisting of HCV protease inhibitors, HCV NS5A inhibitors and HCV NS5B polymerase inhibitors.

15. (canceled)

16. A method of treating a patient infected with HCV comprising the step of administering an amount of the compound according to claim 1, or a pharmaceutically acceptable salt thereof, effective to treat infection by HCV in said patient.

17. The method according to claim 16, further comprising the step of administering pegylated-interferon alpha and ribavirin to said patient.

18. The method according to claim 16, further comprising administering a second therapeutic agent selected from the group consisting of HCV antiviral agents, immunomodulators, and anti-infective agents.

19. The method according to claim 18, further comprising administering a third therapeutic agent selected from the group consisting of HCV protease inhibitors, HCV NS5A inhibitors and HCV NS5B polymerase inhibitors.
